(19) United States
(12) Patent Application Publication
(10) Pub. No.: US 2015/0157539 A1
(43) Pub. Date: Jun. 11, 2015

(54) COSMETIC COMPOSITION COMPRISING COMPOSITE PARTICLES

(71) Applicants: Momoko Shimizu, Kawasaki-shi (JP); Shinichi Matsufuji, Kawasaki (JP); Romain Tachon, Kawasaki (JP)

(72) Inventors: Momoko Shimizu, Kawasaki-shi (JP); Shinichi Matsufuji, Kawasaki (JP); Romain Tachon, Kawasaki (JP)

(73) Assignee: L'OREAL, Paris (FR)

(21) Appl. No.: 14/412,777

(22) PCT Filed: Jul. 13, 2012
(86) PCT No.: PCT/JP2012/068524

§ 371 Date: Jan. 5, 2015

(51) Int. Cl. A61K 8/02; A61K 8/25; A61K 8/29; A61Q 17/04

(52) U.S. Cl. CPC A61K 8/0241; A61Q 17/04; A61K 8/25; A61K 8/29

(57) ABSTRACT

The present invention relates to a cosmetic composition comprising, at least: (i) at least one composite pigment comprising: at least one small particle with a mean particle size of more than 100 nm and less than 1 um, preferably less than 600 nm, and more preferably less than 400 nm, wherein the surface of the small particle is at least in part covered with at least one coating layer comprising at least one inorganic or organic solid UV filter, and optionally at least one coloring agent, preferably at least one coating layer comprising at least one inorganic or organic solid UV filter, and more preferably at least one inorganic solid UV filter; and (ii) at least one plate-type filler with a refractive index of more than 1.6 and less than 2.2 in an amount of more than 1% by weight relative to the total weight of composition.

### TECHNICAL FIELD

The present invention relates to a cosmetic composition comprising a combination of at least one specific composite pigment, and at least one specific plate-type filler in a specific amount condition.

### BACKGROUND ART

Generally, UV protection can be achieved by the introduction of organic or inorganic UV filter(s) into a cosmetic composition. Organic UV filters may provide strong UVA and UVB protection but bring inappropriate level of shine to the cosmetic composition. Inorganic UV filters such as TiO₂ and ZnO pigments have good light scattering properties but inappropriate sensory profile.

Users of cosmetic products tend to expect color homogeneity without irregular colors, and optical effects such as brightening effects for the skin (skin tone improvement).

In conventional cosmetic products, for example, for protecting the skin from UV, a large amount of inorganic fillers such as TiO₂ may be used, and this will make the color of the skin too whitish, and the users of the cosmetic products may feel uncomfortable for the skin color heterogeneity.

In order to enhance the optical effects for the skin, pearlescent pigments such as TiO₂-coated mica may be used in conventional cosmetic products. However, the pearlescent agents make the surface roughness of the skin outstanding, and show color heterogeneity. Thus, the use of a large amount of pearlescent pigments makes unnatural make-up result.

In addition, the users of cosmetic products also expect the stability of the cosmetic products. For example, the cosmetic composition in the cosmetic products should be kept homogeneous for a long time, and should not separate into two or more phases.

### DISCLOSURE OF INVENTION

An objective of the present invention is to provide a cosmetic composition with improved optical cosmetic effects such as skin brightening and color homogeneity, which can also have improved UV filtering effects and stability, without deteriorating other cosmetic effects such as smoothening effects.

The above objective can be achieved by a cosmetic composition comprising:
(i) at least one composite pigment comprising:
   at least one small particle with a mean particle size of more than 100 nm and less than 1 μm, preferably less than 600 nm, and more preferably less than 400 nm, wherein the surface of the small particle is at least in part covered with at least one coating layer comprising at least one inorganic or organic particulate solid UV filter, and optionally at least one coloring pigment, preferably at least one coating layer comprising at least one inorganic or organic particulate solid UV filter, and more preferably at least one inorganic solid UV filter; and
(ii) at least one plate-type filler with a refractive index of more than 1.6 and less than 2.2 in an amount of more than 1% by weight relative to the total weight of composition.

The small particle may be a small hollow particle.

The composite pigment may further comprise at least one large particle with a mean particle size of 2 μm or more, preferably 3 μm or more, more preferably 4 μm or more, and even more preferably 5 μm or more, wherein the surface of the large particle is optionally at least in part covered with at least one coating layer comprising at least one inorganic or organic particulate solid UV filter and/or at least one coloring pigment.

The coating layer on the small and/or large particle(s) may have a thickness of from 1 nm to 50 nm, preferably from 5 nm to 40 nm, and more preferably from 10 nm to 30 nm.

The inorganic solid UV filter may be selected from the group consisting of silicon carbide, metal oxides, and mixtures thereof.

The inorganic solid UV filter may have a mean particle size of from 1 nm to 50 nm, preferably from 5 nm to 40 nm, and more preferably from 10 nm to 30 nm.

It is preferable that the inorganic solid UV filter be titanium dioxide.

The small particle or the large particle may comprise at least one inorganic material and/or at least one organic material, preferably at least one organic material advantageously selected from the group consisting of poly(meth)acrylates, polyamides, silicones, polyurethanes, polyethylenes, polypropylenes, polystyrenes, copolystyrenes, polyhydroxyalkanoates, polycaprolactams, poly(butylene) succinates, polysaccharides, polypeptides, polyvinyl alcohols, polyvinyl resins, fluoropolymers, wax, amidosulfonic acid polyvalent metal salts, acylated amino acids, and mixtures thereof.

As the organic material, the small particle may preferably contain at least one organic polymer. In particular, as the organic polymer, copolystyrene is preferable, and styrene/acrylate copolymer, and cross-linked styrene/methyl methacrylate copolymer are more preferable.

The small particle may comprise at least one copolystyrene, preferably a styrene/acrylate copolymer, and/or a cross-linked styrene/methyl methacrylate copolymer;

the large particle may comprise at least one poly(meth)acrylate, preferably a methyl methacrylate polymer; and
the small and large particles may be at least in part covered with at least one coating layer comprising metal oxide, preferably titanium oxide.

The composite pigment based on a small particle can be obtained by subjecting:

at least one small particle with a mean particle size more than 100 nm and of less than 1 μm, preferably less than 600 nm, and more preferably less than 400 nm;
at least one inorganic or organic particulate solid UV filter; and
optionally at least one coloring pigment and/or at least one additional UV filter to a mechanochemical fusion process.

The composite pigment based on small and large particles can be obtained by subjecting:

at least one small particle with a mean particle size more than 100 nm and of less than 1 μm, preferably less than 600 nm, and more preferably less than 400 nm;
at least one large particle with a mean particle size of 2 μm or more, preferably 3 μm or more, more preferably 4 μm or more, and even more preferably 5 μm or more;
at least one inorganic or organic particulate solid UV filter; and
optionally at least one coloring pigment and/or at least one additional UV filter to a mechanochemical fusion process.

The plate-type filler may be present in the composition in an amount ranging from 1% to 20% by weight of the cosmetic composition according to the present invention, and preferably from 2% to 10% by weight relative to the total weight of the composition.

The plate-type filler may be selected from the group consisting of boron nitride, barium sulfate, bismuth oxychloride, alumina and composite powders based on titanium oxide and substrate like talc, mica, barium sulfate, boron nitride, bismuth oxychloride, alumina and mixtures thereof.

The plate-type filler may have a particle size from 1 to 15 μm, preferably from 1 to 10 μm.

The cosmetic composition according to the present invention may be in the form of a liquid, powder or aerosol foam.

In a particular embodiment, the cosmetic composition of the present invention is a base or a primer, in particular a skin care or a make-up base or primer.

In a particular embodiment, the cosmetic composition of the present invention comprises a low amount of additional coloring pigments, as a high amount of additional coloring pigments may alter the brightening effect.

The term ‘additional coloring pigments’ according to the present invention means here additional coloring pigments used as ingredients dispersed in the cosmetic composition according to the present invention, which are distinct from the coloring pigments that may be present in the coating of the said composite pigments.

Then, the cosmetic composition according to the present invention may comprise from 0 to 5% of additional coloring pigments relative to the total weight of the composition.

In a particular embodiment, the cosmetic composition according to the present invention comprises from 0 to 3% of additional coloring pigments relative to the total weight of the composition.

Another objective of the present invention is to provide a cosmetic process with advantageous cosmetic and/or practical effects by using the cosmetic composition according to the present invention.

The above objective can be achieved by applying the cosmetic composition according to the present invention onto the skin.

Thus, the present invention also relates to a cosmetic process for improving optical brightness and/or homogeneity of the skin, comprising the application on the skin of at least one layer of the cosmetic composition according to the present invention.

### BEST MODE FOR CARRYING OUT THE INVENTION

After diligent research, the inventors have discovered that it is possible to provide a cosmetic composition with improved optical cosmetic effects such as skin brightening and color homogeneity, which can also have improved UV filtering effects and stability, without deteriorating other cosmetic effects such as smoothening effects.

Thus, the cosmetic composition according to the present invention comprises, at least:
(i) at least one composite pigment comprising:
   at least one small particle with a mean particle size of more than 100 nm and less than 1 μm, preferably less than 600 nm, and more preferably less than 400 nm, wherein the surface of the small particle is at least in part covered with at least one coating layer comprising at least one inorganic or organic particulate solid UV filter, and optionally at least one coloring agent, preferably at least one coating layer comprising at least one inorganic or organic particulate solid UV filter, and more preferably at least one inorganic solid UV filter; and
(ii) at least one plate-type filler with a refractive index of more than 1.6 and less than 2.2 in an amount of more than 1% by weight relative to the total weight of composition.

It is preferable that the composite pigment further comprise at least one large particle with a mean particle size of 2 μm or more, preferably 3 μm or more, more preferably 4 μm or more, and even more preferably 5 μm or more, wherein the surface of the large particle is optionally at least in part covered with at least one coating layer comprising at least one inorganic or organic particulate solid UV filter and/or at least one coloring pigment.

This cosmetic composition according to the present invention can have increased stability so that it can be used for a long time without, for example, phase separation. Furthermore, the cosmetic composition according to the present invention can provide skin brightness and/or homogeneity of the skin, in other words, bright skin look and/or homogeneous skin color, as well as improved UV protection, without deteriorating other cosmetic properties such as smooth feeling to touch.

Hereafter, each of the elements constituting the cosmetic composition according to the present invention will be described in a detailed manner.

### [Composite Pigment]

The cosmetic composition according to the present invention includes at least one specific composite pigment comprising, at least, at least one small particle wherein the surface of the small particle is at least in part covered with at least one coating layer comprising at least one inorganic or organic particulate solid UV filter.

### (Small Core Particle)

The small core particle for the composite pigment used in the present invention is not limited, as long as the small core particle has a mean particle size or a mean particle diameter of more than 100 nm and less than 1 μm, preferably less than 600 nm, and more preferably less than 400 nm.

The small core particle may be in the form of a solid or hollow particle, preferably a hollow particle.

The mean particle size or mean particle diameter here is an arithmetric mean diameter, and can be determined, for example, by calculating the average of the dimensions of one hundred particles chosen on an image obtained with a scanning electron microscope.

The small core particle can be in any shape. For example, it is possible to use a small core particle in the form of a plate with an aspect ratio of at least 5, preferably more than 10, more preferably more than 20, and more preferably more than 50. The aspect ratio can be determined by the average thickness and the average length according to the formula: aspect ratio=length/thickness.

If a plate-like particle is used for the present invention, it is preferable that the plate-like particle have a length ranging from more than 100 nm to less than 1 μm, preferably less than 600 nm, and more preferably less than 400 nm.

In a preferred embodiment, the small core particle has a spherical shape.

The material of the small core particle is not limited. The material can be at least one inorganic material and/or at least one organic material, preferably at least one organic material.

The inorganic material and/or organic material may be porous. The porosity of the material may be characterized by a specific surface area of from 0.05 m²/g to 1,500 m²/g, more preferably from 0.1 m²/g to 1,000 m²/g, and more preferably from 0.2 m²/g to 500 m²/g according to the BET method.

Preferably, the inorganic material can be selected from the group consisting of mica, synthetic mica, talc, sericite, boron nitride, glass flakes, calcium carbonate, barium sulfate, titanium oxide, hydroxyapatite, silica, silicate, zinc oxide, magnesium sulfate, magnesium carbonate, magnesium trisilicate, aluminum oxide, aluminum silicate, calcium silicate, calcium phosphate, magnesium oxide, bismuth oxychloride, kaolin, hydrotalcite, mineral clay, synthetic clay, iron oxide, and mixtures thereof. In particular, natural mica, synthetic mica, sericite, kaolin, talc and mixtures thereof are preferable.

Preferably, the organic material can be selected from the group consisting of poly(meth)acrylates, polyamides, silicones, polyurethanes, polyethylenes, polypropylenes, polystyrenes, copolystyrenes, polyhydroxyalkanoates, polycaprolactams, poly(butylene) succinates, polysaccharides, polypeptides, polyvinyl alcohols, polyvinyl resins, fluoropolymers, waxes, amidosulfonic acid polyvalent metal salts, acylated amino acids, and mixtures thereof. As fluoropolymers, for example, PTFE may be used. As amidosulfonic acid polyvalent metal salts, for example, N-lauroyltaurine calcium may be used. As acylated amino acids, lauroyllysine may be used. Polyamides such as Nylon®, polyhydroxyalkanoates such as polylactic acids, poly(meth)acrylates such as polymethylmethacrylates, silicones, and mixtures thereof are more preferable.

In particular, as the organic material, copolystyrene is preferable, and styrene/acrylate copolymer, and cross-linked styrene/methyl methacrylate copolymer are more preferable. Thus, as the small core particles, for example, Sunspheres (small hollow particles made from styrene/acrylate copolymer) marketed by Rohm and Hans, as well as SX859(A) and SX866(B) (small hollow particles made from cross-linked styrene/methyl methacrylate copolymer) marketed by JSR Corp. in Japan, are preferable. In addition, polymethylmethacrylate solid small particles such as MP2200 marketed by Soken in Japan are also preferable as organic small core particles.

The small core particle may or may not be coated beforehand.

In a particular embodiment, the small core particle is originally coated. The material of an original coating of the small core particle is not limited, but an organic material such as an amino acid, an N-acylamino acid, an amido, a silicone and a modified silicone, may be preferable. As the organic material, mention may be made of lauroyl lysine and acryl-modified silicone.

### (Layer on Small Core Particle)

The small core particle is at least partially covered with at least one layer comprising at least one inorganic or organic particulate solid UV filter, preferably at least one inorganic solid UV filter. The layer may be referred to as a coating layer. Preferably, 10% or more of the surface of the small core particle can be covered by the coating layer(s). More preferably, 50% or more of the surface of the small core particle can be covered by the coating layer(s). More preferably, 80% or more of the small core particle can be covered by the coating layer(s). Most preferably, the entire surface of the small core particle can be covered by the coating layer(s).

The thickness of the coating layer may vary depending on several factors such as the size of the small core particle. Typically, the thickness of the coating layer may range from 1 nm to 50 nm, preferably from 5 nm to 40 nm, and more preferably from 10 nm to 30 nm.

If there are two or more coating layers on the small core particle, the thickness and the composition of the coating layers may be the same as or different from each other.

The coating layer(s) may comprise, other than the inorganic or organic particulate solid UV filter(s), any additional material(s) such as coloring pigment(s) and/or additional UV filter(s), preferably liquid UV filter(s). The additional material(s) may be present in an amount ranging from 1 to 50 wt % relative to the total weight of the additional material(s) and the inorganic or organic particulate solid UV filter(s).

### (Inorganic Solid UV Filter)

As described above, the coating layer(s) on the small core particle may comprise at least one inorganic solid UV filter. If two or more inorganic solid UV filters are used, they may be the same or different, preferably the same.

The inorganic solid UV filter used for the present invention may be active in the UV-A and/or UVB region, preferably in the UVB region or in the UV-A and UVB region. It is preferable that the active UV filtering region of the inorganic solid UV filter and that of the particulate organic solid UV filter be complementary to each other, in order to provide comprehensive UV protection. For example, it is preferable that the inorganic solid UV filter be active at least in the UVB region and the particulate organic solid UV filter be active at least in the UV-A region. The inorganic solid UV filter may be hydrophilic and/or lipophilic. The inorganic solid UV filter is completely insoluble in solvents such as water and ethanol commonly used in cosmetics. The term “solid” means solid at 25° C. under 1 atm.

It is preferable that the inorganic solid UV filter be in the form of a fine particle such that the mean (primary) particle diameter thereof ranges from 1 nm to 50 nm, preferably from 5 nm to 40 nm, and more preferably from 10 nm to 30 nm. The mean (primary) particle size or mean (primary) particle diameter here is an arithmetric mean diameter.

The inorganic solid UV filter may be selected from the group consisting of silicon carbide, metal oxides which may or may not be coated, and mixtures thereof.

Preferably, the inorganic solid UV filters are selected from pigments (mean size of the primary particles: generally from 5 nm to 50 nm, preferably from 10 nm to 50 nm) formed of metal oxides, such as, for example, pigments formed of titanium oxide (amorphous or crystalline in the rutile and/or anatase form), iron oxide, zinc oxide, zirconium oxide or cerium oxide, which are all UV photoprotective agents well known per se. Preferably, the inorganic solid UV filters are selected from titanium oxide, zinc oxide, and more preferably titanium oxide.

The inorganic solid UV filter may or may not be coated. The inorganic solid UV filter may have at least one coating. The coating may comprise at least one compound selected from the group consisting of alumina, silica, aluminum hydroxide, silicones, silanes, fatty acids or salts thereof (such as sodium, potassium, zinc, iron or aluminum salts), fatty alcohols, lecithin, amino acids, polysaccharides, proteins, alkanolamines, waxes such as beeswax, (meth)acrylic polymers, organic UV filters, and (per)fluoro compounds.

It is preferable for the coating to include at least one organic UV filter. As the organic UV filter in the coating, a dibenzoylmethane derivative such as butyl methoxydibenzoylmethane (Avobenzone) and 2,2′-Methylenebis[6-(2H-Benzotriazol-2-yl)-4-(1,1,3,3-Tetramethyl-Butyl)Phenol](Methylene Bis-Benzotriazolyl Tetramethylbutylphenol) marketed as “TINOSORB M” by BASF may be preferable.

In a known manner, the silicones in the coating(s) may be organosilicon polymers or oligomers comprising a linear or cyclic and branched or cross-linked structure, of variable molecular weight, obtained by polymerization and/or polycondensation of suitable functional silanes and essentially composed of a repetition of main units in which the silicon atoms are connected to one another via oxygen atoms (siloxane bond), optionally substituted hydrocarbon radicals being connected directly to the said silicon atoms via a carbon atom.

The term “silicones” also encompasses silanes necessary for their preparation, in particular alkylsilanes.

The silicones used for the coating(s) can preferably be selected from the group consisting of alkylsilanes, polydialkylsiloxanes and polyalkylhydrosiloxanes. More preferably still, the silicones are selected from the group consisting of octyltrimethylsilane, polydimethylsiloxanes and polymethylhydrosiloxanes.

Of course, the inorganic solid UV filters made of metal oxides may, before their treatment with silicones, have been treated with other surfacing agents, in particular with cerium oxide, alumina, silica, aluminum compounds, silicon compounds or their mixtures.

The coated inorganic solid UV filter may have been prepared by subjecting the inorganic solid UV filter to one or more surface treatments of a chemical, electronic, mechanochemical and/or mechanical nature with any of the compounds as described above, as well as polyethylenes, metal alkoxides (titanium or aluminum alkoxides), metal oxides, sodium hexametaphosphate, and those shown, for example, in Cosmetics & Toiletries, February 1990, Vol. 105, pp. 53-64.

The coated inorganic solid UV filters may be titanium oxides coated:

with silica, such as the product “Sunveil” from Ikeda;
with silica and with iron oxide, such as the product “Sunveil F” from Ikeda;
with silica and with alumina, such as the products “Microtitanium Dioxide MT 500 SA” from Tayca, “Tioveil” from Tioxide, and “Mirasun TiW 60” from Rhodia;
with alumina, such as the products “Tipaque TTO-55 (B)” and “Tipaque TTO-55 (A)” from Ishihara, and “UVT 14/4” from Kemira;
with alumina and with aluminum stearate, such as the product “Microtitanium Dioxide MT 100 T, MT 100 TX, MT 100 Z or MT-01” from Tayca, the products “Solaveil CT-10 W” and “Solaveil CT 100” from Uniqema, and the product “Eusolex T-AVO” from Merck;
with alumina and with aluminum laurate, such as the product “Microtitanium Dioxide MT 100 S” from Tayca;
with iron oxide and with iron stearate, such as the product “Microtitanium Dioxide MT 100 F” from Tayca;
with zinc oxide and with zinc stearate, such as the product “BR351” from Tayca;
with silica and with alumina and treated with a silicone, such as the products “Microtitanium Dioxide MT 600 SAS”, “Microtitanium Dioxide MT 500 SAS” and “Microtitanium Dioxide MT 100 SAS” from Tayca;
with silica, with alumina and with aluminum stearate and treated with a silicone, such as the product “STT-30-DS” from Titan Kogyo;
with silica and treated with a silicone, such as the product “UV-Titan X 195” from Kemira;
with alumina and treated with a silicone, such as the products “Tipaque TTO-55 (S)” from Ishihara or “UV Titan M 262” from Kemira;
with triethanolamine, such as the product “STT-65-S” from Titan Kogyo;
with stearic acid, such as the product “Tipaque TTO-55 (C)” from Ishihara; or
   with sodium hexametaphosphate, such as the product “Microtitanium Dioxide MT 150 W” from Tayca.

Other titanium oxide pigments treated with a silicone are preferably TiO₂ treated with octyltrimethylsilane and for which the mean size of the individual particles is from 25 and 40 nm, such as that marketed under the trademark “T 805” by Degussa Silices, TiO₂ treated with a polydimethylsiloxane and for which the mean size of the individual particles is 21 nm, such as that marketed under the trademark “70250 Cardre UF TiO₂Si₃” by Cardre, anatase/rutile TiO₂ treated with a polydimethylhydrosiloxane and for which the mean size of the individual particles is 25 nm, such as that marketed under the trademark “Microtitanium Dioxide USP Grade Hydrophobic” by Color Techniques.

Preferably, the following coated TiO₂ can be used as the coated inorganic UV filter: Stearic acid (and) Aluminum Hydroxide (and) TiO₂, such as the product “MT-100 TV” from Tayca, with a mean primary particle diameter of 15 nm;

Dimethicone (and) Stearic Acid (and) Aluminum Hydroxide (and) TiO₂, such as the product “SA-TTO-S4” from Miyoshi Kasei, with a mean primary particle diameter of 15 nm;
Silica (and) TiO₂, such as the product “MT-100 WP” from Tayca, with a mean primary particle diameter of 15 nm;
Dimethicone (and) Silica (and) Aluminum Hydroxide (and) TiO₂, such as the product “MT-Y02” and “MT-Y-110 M3S” from Tayca, with a mean primary particle diameter of 10 nm;
Dimethicone (and) Aluminum Hydroxide (and) TiO₂, such as the product “SA-TTO-S3” from Miyoshi Kasei, with a mean primary particle diameter of 15 nm;
Dimethicone (and) Alumina (and) TiO₂, such as the product “UV TITAN M170” from Sachtleben, with a mean primary particle diameter of 15 nm; and
Silica (and) Aluminum Hydroxide (and) Alginic Acid (and) TiO₂, such as the product “MT-100 AQ” from Tayca, with a mean primary particle diameter of 15 nm.

In terms of UV filtering ability, TiO₂ coated with at least one organic UV filter is more preferable. For example, Avobenzone (and) Stearic Acid (and) Aluminum Hydroxide (and) TiO₂, such as the product “HXMT-100ZA” from Tayca, with a mean primary particle diameter of 15 nm, can be used.

The uncoated titanium oxide pigments are, for example, marketed by Tayca under the trademarks “Microtitanium Dioxide MT500B” or “Microtitanium Dioxide MT600B”, by Degussa under the trademark “P 25”, by Wacker under the trademark “Oxyde de titane transparent PW”, by Miyoshi Kasei under the trademark “UFTR”, by Tomen under the trademark “ITS” and by Tioxide under the trademark “Tioveil AQ”.

The uncoated zinc oxide pigments are, for example:

those marketed under the trademark “Z-cote” by Sunsmart;
those marketed under the trademark “Nanox” by Elementis; and
those marketed under the trademark “Nanogard WCD 2025” by Nanophase Technologies.

The coated zinc oxide pigments are, for example:

those marketed under the trademark “Oxide Zinc CS-5” by Toshiba (ZnO coated with polymethylhydrosiloxane);
those marketed under the trademark “Nanogard Zinc Oxide FN” by Nanophase Technologies (as a 40% dispersion in Finsolv TN, C₁₂-C₁₅ alkyl benzoate);
those marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nano-oxides coated with silica and polymethylhydrosiloxane);
those marketed under the trademark “NFD Ultrafine ZnO” by Daikin (ZnO coated with phosphate of perfluoroalkyl and a copolymer based on perfluoroalkylethyl as a dispersion in cyclopentasiloxane);
those marketed under the trademark “SPD-Z1” by Shin-Etsu (ZnO coated with a silicone-grafted acrylic polymer dispersed in cyclodimethylsiloxane);
those marketed under the trademark “Escalol Z100” by ISP (alumina-treated ZnO dispersed in an ethylhexyl methoxycinnamate/PVP-hexadecene copolymer/methicone mixture); and
those marketed under the trademark “Fuji ZnO-SMS-10” by Fuji Pigment (ZnO coated with silica and polymethylsilsesquioxane); those marketed under the trademark “Nanox Gel TN” by Elementis (ZnO dispersed at 55% in C₁₂-C₁₅ alkyl benzoate with hydroxystearic acid polycondensate).

The uncoated cerium oxide pigments are marketed, for example, under the trademark “Colloidal Cerium Oxide” by Rhone-Poulenc.

The uncoated iron oxide pigments are, for example, marketed by Arnaud under the trademarks “Nanogard WCD 2002 (FE 45B)”, “Nanogard Iron FE 45 BL AQ”, “Nanogard FE 45R AQ” and “Nanogard WCD 2006 (FE 45R)”, or by Mitsubishi under the trademark “TY-220”.

The coated iron oxide pigments are, for example, marketed by Arnaud under the trademarks “Nanogard WCD 2008 (FE 45B FN)”, “Nanogard WCD 2009 (FE 45B 556)”, “Nanogard FE 45 BL 345” and “Nanogard FE 45 BL” or by BASF under the trademark “Oxyde de fer transparent”.

Mention may also be made of mixtures of metal oxides, in particular of titanium dioxide and of cerium dioxide, including a mixture of equal weights of titanium dioxide coated with silica and of cerium dioxide coated with silica marketed by Ikeda under the trademark “Sunveil A”, and also a mixture of titanium dioxide and of zinc dioxide coated with alumina, with silica and with silicone, such as the product “M 261” marketed by Kemira, or coated with alumina, with silica and with glycerol, such as the product “M 211” marketed by Kemira.

Coated inorganic solid UV filters are preferable, because the UV filtering effects of the inorganic solid UV filters can be enhanced. In addition, the coating(s) may function as a binder for fixing the UV filters on a small core particle.

If the inorganic solid UV filter(s) in the form of fine particles is/are used, the composite pigment used in the present invention has an effect of not providing a white appearance but a transparent or clear appearance, because the fine particles of the inorganic solid UV filters do not aggregate but spread on the core particle. It should be noted that free fine particles of inorganic solid UV filter(s) easily aggregate to give a white appearance to the skin.

The inorganic solid UV filter(s) may be used in the composite pigment in proportions such that the weight ratio of the small core particle(s) to the inorganic solid UV filter(s) is from 10:90 to 90:10, preferably from 30:70 to 70:30, and more preferably from 40:60 to 50:50.

### (Particulate Organic Solid UV Filter)

As described above, the coating layer on the small core particle may comprise at least one particulate organic solid UV filter. If two or more particulate organic solid UV filters are used, they may be the same or different, preferably the same. The term “UV filters” may be paraphrased with “UV screening agents”.

The particulate organic solid UV filter used for the present invention may be active in the UV-A and/or UVB region, preferably in the UV-A region or in the UV-A and UVB region. The organic solid UV filter may be hydrophilic and/or lipophilic.

<<Particulate organic solid UV filter>>, means an organic molecule which (1) is under the form of solid particles at 25° C. and insoluble in the medium of the composition of the invention and (2) which allow by absorption, and/or reflection and/or diffusion of the UVA and/or UVB radiations allows to block or at least to limit the contact of the said radiations with the surface of keratinic materials (skin, hair, scalp).

The term “solid” means solid at 25° C. under 1 atm.

The particulate organic solid UV filters used in the present invention have preferentially a mean particle size which varies from 10 to 5 μm and more preferably from 10 nm to 2 μm and more particularly from 20 nm to 2 μm.

The particulate organic solid UV filters used in the present invention can be brought to the desired particulate form by any ad hoc means, such as, in particular, dry milling or milling in a solvent medium, sieving, atomization, micronization or spraying.

An example of a process for the micronization of insoluble particulate organic UV filters is disclosed in Applications GB-A-2303549 and EP-A-893119, which are incorporated by reference to form an integral part of the description. The milling device used according to these documents can be an airjet mill, bead mill, vibration mill or hammer mill and preferably a mill with high-speed stirring or an impact mill and more particularly a rotary bead mill, a vibrating mill, a tube mill or a rod mill.

The composite pigment used in the present invention has an effect that of providing a transparent or clear appearance, because the fine particles of the particulate organic solid UV filter(s) do not aggregate but spread on the core particle. It should be noted that free fine particles of particulate organic solid UV filter(s) can easily aggregate.

The material of the particulate organic solid UV filter(s) is not limited as long as it is organic. If two or more particulate organic solid UV filters are used, the material(s) of the particulate organic solid UV filters may be the same as or different from each other.

The particulate solid organic UV screening agents used in the present invention can be chosen in particular from particulate organic UV screening agents of the oxalanilide type, of the triazine type, of the benzotriazole type; of the vinyl amide type; of the cinnamamide type; of the type comprising one or more benzazole and/or benzofuran or benzothiophene groups or of the indole type; of the aryl vinylene ketone type; of the phenylenebis(benzoxazinone) derivative type; or of the acrylonitrile amide, sulphonamide or carbamate derivative type.

In the sense in which it is used in the present invention, the term benzazole simultaneously encompasses benzothiazoles, benzoxazoles and benzimidazoles.

Mention may be made, among UV screening agents, of the oxalanilide type in accordance with the invention, of those corresponding to the structure:

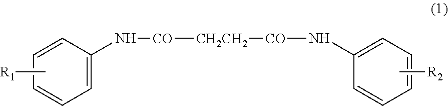

in which R₁ and R₂, independently, are C₁-C₁₈ alkyl or C₁-C₁₈ alkoxy. A preferred compound of formula (1) is N-(2-ethoxyphenyl)-N-(2-ethylphenyl)-ethanediamide. These compounds are disclosed in Patent Application WO 95/22959.

Mention may be made, as examples, of the commercial products Tinuvin 315 and Tinuvin 312, sold by Ciba-Geigy, with the respective structures:

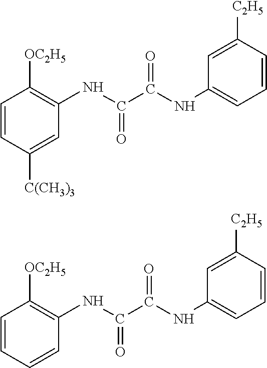 [SMILES: CCOc1ccc(C(C)(C)C)cc1NC(=O)C(=O)Nc1cccc(CC)c1 ; CCOc1ccccc1NC(=O)C(=O)Nc1cccc(CC)c1]

A preferred class of solid triazine UV absorbers is that having the formula:

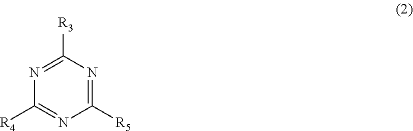

in which R₃, R₄ and R₅, independently, are H, OH, C₁-C₁₈ alkoxy, NH₂, NH—R₆ or N(R₆)₂ in which R₆ is C₁-C₁₈ alkyl, OR₆ in which R₆ is C₁-C₁₈ alkyl, phenyl, phenoxy or anilino, or pyrrole, in which the respective phenyl, phenoxy or anilino, or pyrrolo moieties are optionally substituted by one, two or three substituents selected from OH, carboxy, CO—NH₂, C₁-C₁₈ alkyl or alkoxy, C₁-C₁₈ carboxyalkyl, C₅-C₈ cycloalkyl, a methylidenecamphor group, the group —(CH═CH)ₘC(═O)—OR₆ in which m is 0 or 1 and R₆ has the same meaning as above, or the group

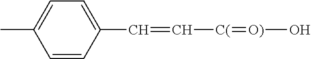

or the corresponding alkali metal, ammonium, mono-, di- or tri-C₁-C₄ alkylammonium, mono-, di- or tri-C₂-C₄ alkanolammonium salts, or the C₁-C₁₈ alkyl esters thereof.

These compounds are disclosed in WO 97/03642, GB 2286774, EP 743309, WO 98/22447 and GB 2319523 (which are incorporated by reference as an integral part of the content of the description).

Preferred compounds of formula (2) are those having one of the formulae:

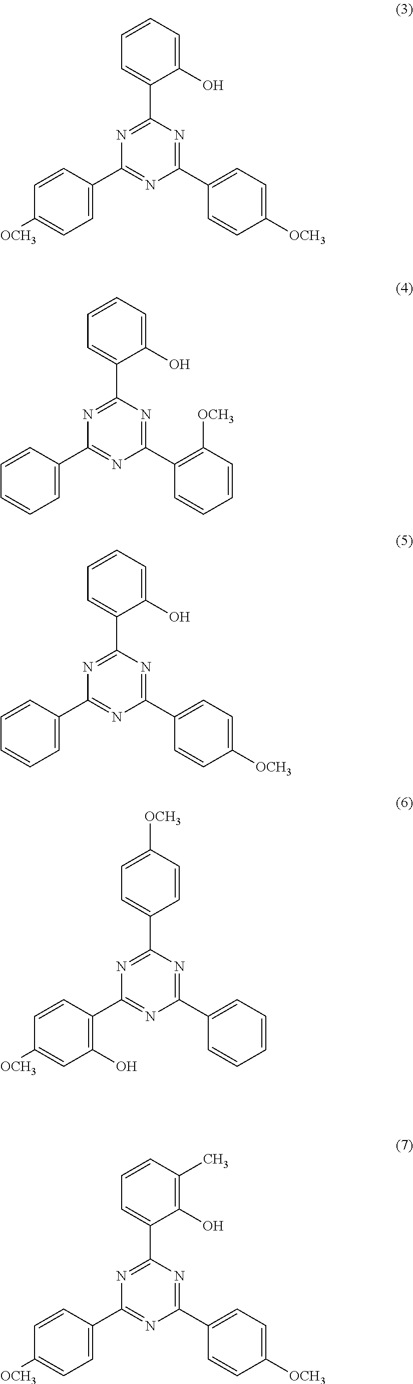 [SMILES: COc1ccc(-c2nc(-c3ccc(OC)cc3)nc(-c3ccccc3O)n2)cc1 ; COc1ccccc1-c1nc(-c2ccccc2)nc(-c2ccccc2O)n1 ; COc1ccc(-c2nc(-c3ccccc3)nc(-c3ccccc3O)n2)cc1 ; COc1ccc(-c2nc(-c3ccccc3)nc(-c3ccc(OC)cc3O)n2)cc1 ; COc1ccc(-c2nc(-c3ccc(OC)cc3)nc(-c3cccc(C)c3O)n2)cc1]

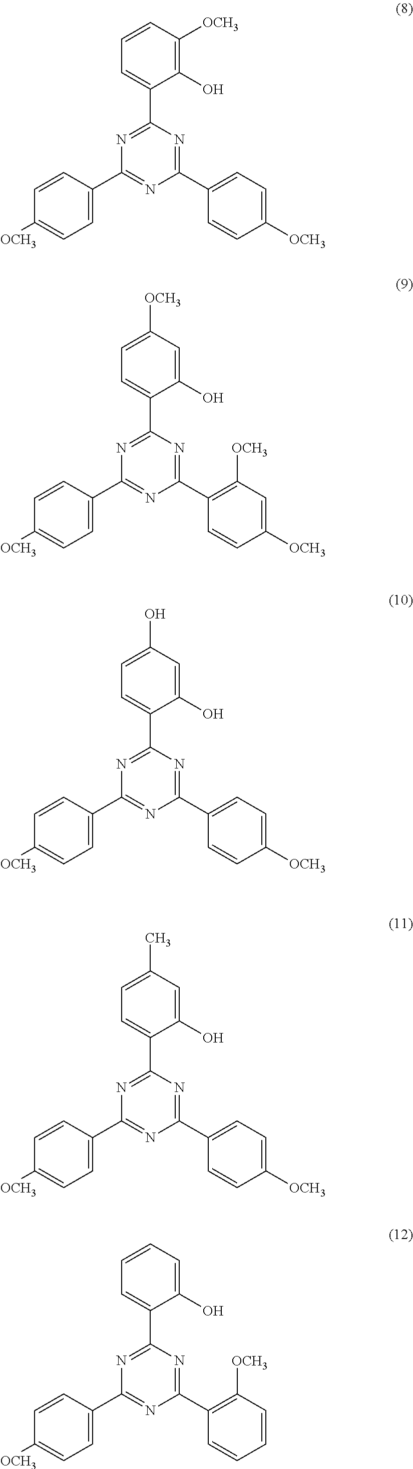 [SMILES: COc1ccc(-c2nc(-c3ccc(OC)cc3)nc(-c3cccc(OC)c3O)n2)cc1 ; COc1ccc(-c2nc(-c3ccc(OC)cc3O)nc(-c3ccc(OC)cc3OC)n2)cc1 ; COc1ccc(-c2nc(-c3ccc(OC)cc3)nc(-c3ccc(O)cc3O)n2)cc1 ; COc1ccc(-c2nc(-c3ccc(OC)cc3)nc(-c3ccc(C)cc3O)n2)cc1 ; COc1ccc(-c2nc(-c3ccccc3O)nc(-c3ccccc3OC)n2)cc1]

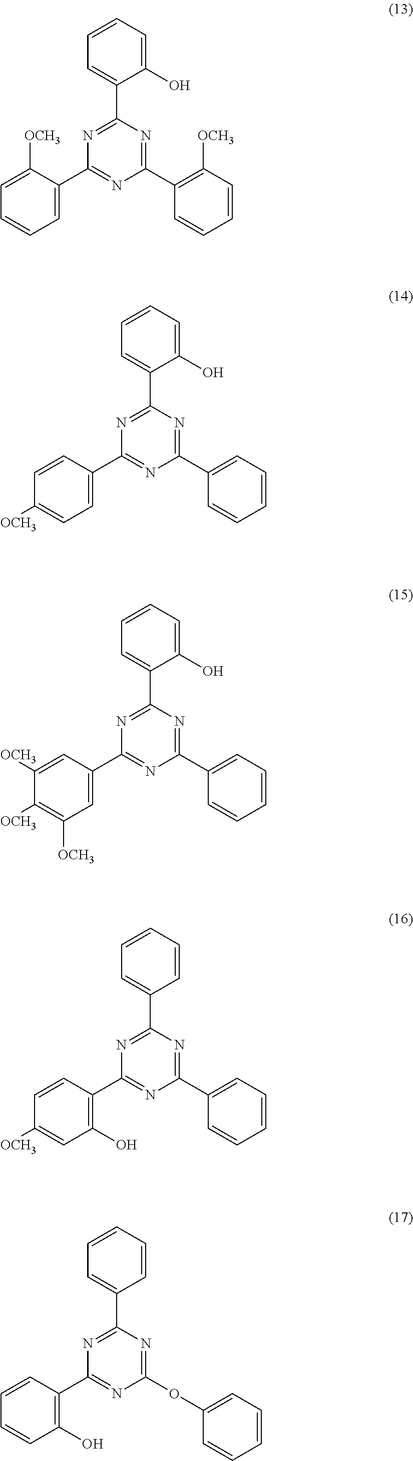 [SMILES: COc1ccccc1-c1nc(-c2ccccc2O)nc(-c2ccccc2OC)n1 ; COc1ccc(-c2nc(-c3ccccc3)nc(-c3ccccc3O)n2)cc1 ; COc1cc(-c2nc(-c3ccccc3)nc(-c3ccccc3O)n2)cc(OC)c1OC ; COc1ccc(-c2nc(-c3ccccc3)nc(-c3ccccc3)n2)c(O)c1 ; Oc1ccccc1-c1nc(Oc2ccccc2)nc(-c2ccccc2)n1]

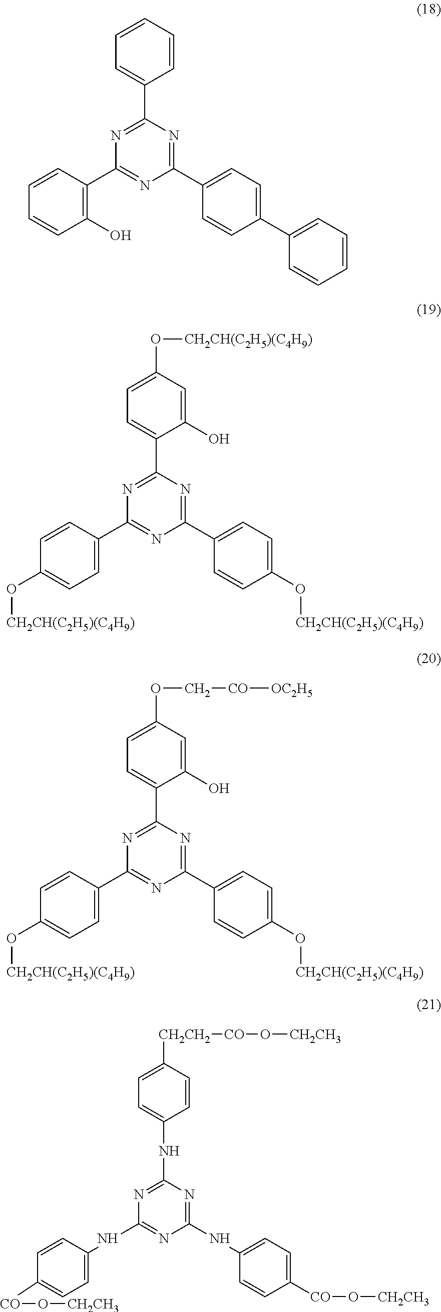

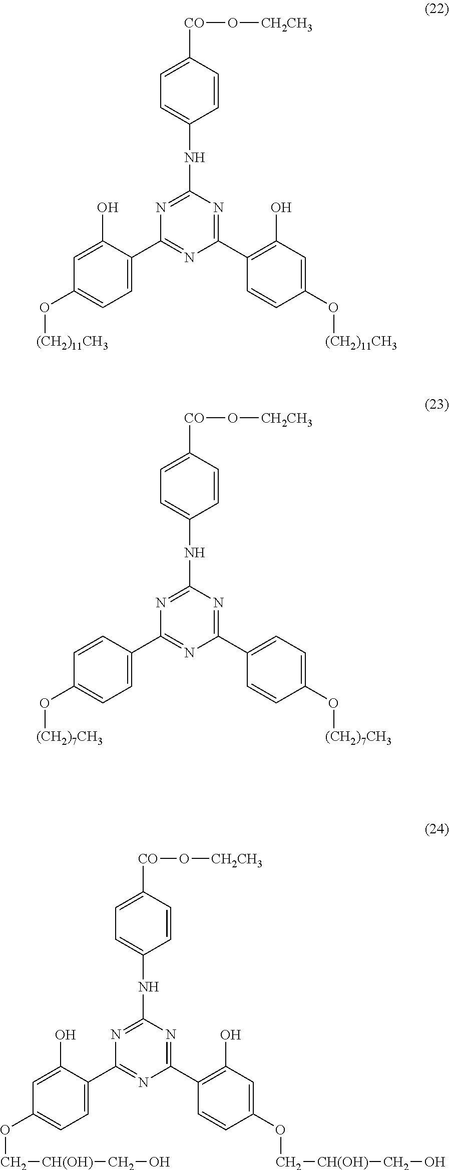

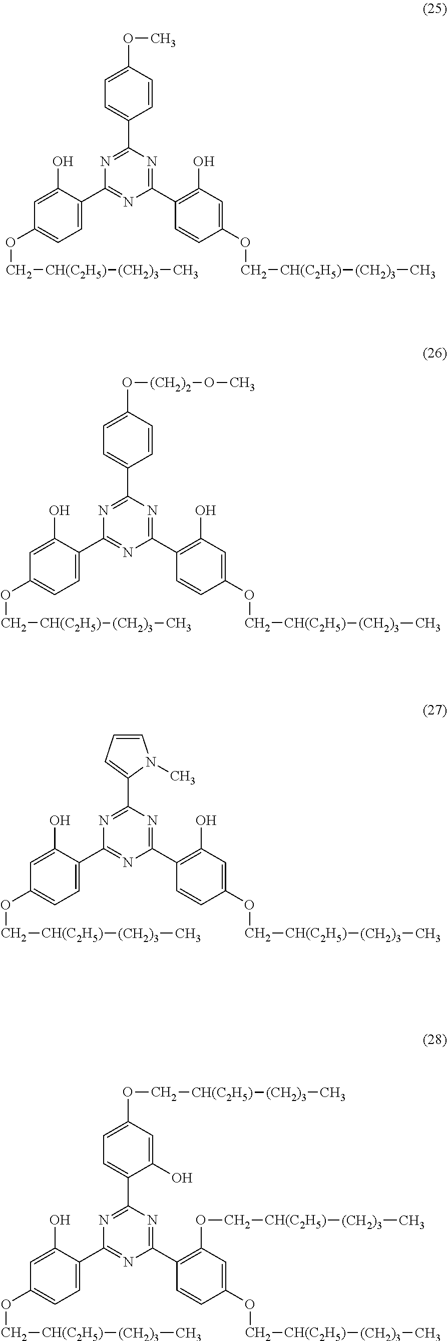

and
as well as 2,4,6-tris(diisobutyl-4′-aminobenzalmalonate)-s-triazine and 2,4-bis(diisobutyl-4-aminobenzalmalonate)-6-(4′-aminobenzylidenecamphor)-s-triazine.

Bis-ethylhexyloxyphenol methoxyphenyl triazine, marketed under the trademark “Tinosorb S” by Ciba-Geigy is in particular preferable.

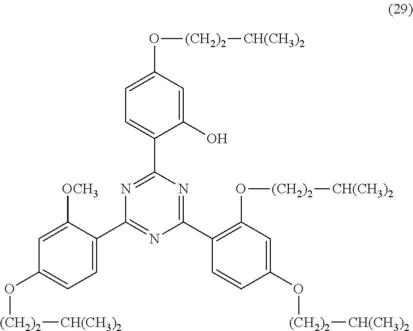

Particularly preferred compounds of formula (2) are those having the formula:

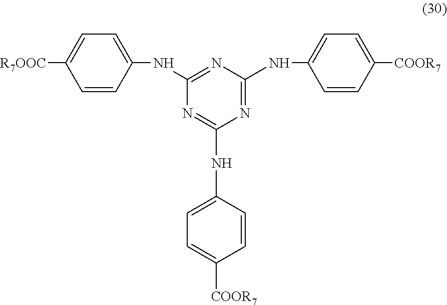

in which the individual radicals R₇ are the same or different and each is hydrogen; an alkali metal; an ammonium group N(R₈)₄ in which R₈ is hydrogen or an organic radical; C₁-C₂₀ alkyl; or a polyoxyethylene radical which contains from 1 to 10 ethylene oxide units and the terminal OH group of which may be etherified by a C₁-C₃ alcohol.

In relation to the compounds of formula (30), when R₇ is an alkali metal it is preferably potassium or, especially sodium; when R₇ is the group N(R₈)₄ in which R₈ has its previous meaning, it is preferably a mono-, di- or tri-C₁-C₄ alkylammonium salt, a mono-, di- or tri-C₂-C₄ alkanolammonium salt or a C₁-C₂₀ alkyl ester thereof; when R₈ is a C₁-C₂₀ alkyl group, it is preferably a C₆-C₁₂ alkyl group, more preferably a C₈-C₉ alkyl group, especially a 3,5,5-trimethylpentyl group or, most particularly, a 2-ethylhexyl group; and when R₈ is a polyoxyethylene group, this preferably contains from 2-6 ethylene oxide units.

Mention may also be made, among UV screening agents of the triazine type in accordance with the invention, of insoluble s-triazine derivatives carrying benzalmalonate and/or phenylcyanoacrylate groups, such as those disclosed in Application EP-A-0 790 243 (which is incorporated by reference as an integral part of the content of the description).

Mention will more particularly be made, among these insoluble UV screening agents of the triazine type, of the following compounds:
2,4,6-tris(diethyl 4′-aminobenzalmalonate)-s-triazine,
2,4,6-tris(diisopropyl 4′-aminobenzalmalonate)-s-triazine,
2,4,6-tris(dimethyl 4′-aminobenzalmalonate)-s-triazine,
2,4,6-tris(ethyl α-cyano-4-aminocinnamate)-s-triazine.

Mention may also be made, among UV screening agents of the triazine type in accordance with the invention, of insoluble s-triazine derivatives carrying benzotriazole and/or benzothiazole groups, such as those disclosed in Application WO 98/25922 (which is incorporated by reference to forms an integral part of the content of the description).

Mention may more particularly be made, among these compounds, of:
2,4,6-tris[(3′-(benzotriazol-2-yl)-2′-hydroxy-5′-methyl)phenylamino]-s-triazine,
2,4,6-tris[(3′-(benzotriazol-2-yl)-2′-hydroxy-5′-tert-octyl)phenylamino]-s-triazine.

A preferred class of solid (benzo)triazole UV absorbers is that having the formula:

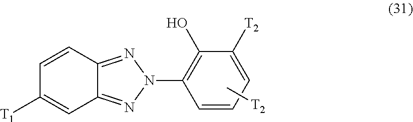

in which T₁ is C₁-C₁₈ alkyl or, preferably, hydrogen; and T₂ is hydrogen, hydroxyl, or C₁-C₁₈ alkyl, optionally substituted by C₁-C₁₂ cycloalkyl or an aryl such as phenyl, preferably α,α-dimethylbenzyl. The C₁-C₁₈ alkyl groups can be linear or branched and are, for example, methyl, ethyl, n-propyl, isopropyl, n-butyl, isobutyl, tert-butyl, tert-octyl, n-amyl, n-hexyl, n-heptyl, n-octyl, isooctyl, n-nonyl, n-decyl, n-undecyl, n-dodecyl, tetradecyl, hexadecyl or octadecyl; the C₅-C₁₂ cycloalkyl groups are, for example, cyclopentyl, cyclohexyl or cyclooctyl; and the aryl groups are, for example, phenyl or benzyl.

Mention may be made, as examples of compounds of formula (31), of the commercial products Tinuvin 328, 320, 234 and 350 from Ciba-Geigy, with the following structures:

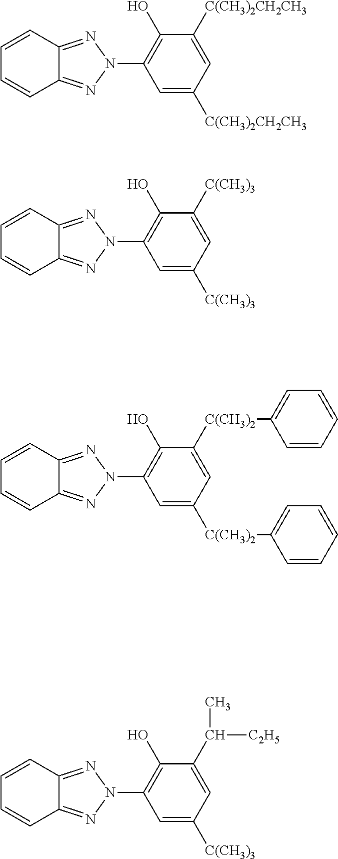

Mention may be made, among insoluble organic UV screening agents of the benzotriazole type in accordance with the invention, of the compounds as disclosed in U.S. Pat. No. 5,687,521, U.S. Pat. No. 5,373,037 and U.S. Pat. No. 5,362,881 and in particular [2,4′-dihydroxy-3-(2H-benzotriazol-2-yl)-5-(1,1,3,3-tetramethylbutyl)-2′-(n-octoxy)-5′-benzoyl]diphenylmethane, sold under the name Mixxim PB30 by Fairmount Chemical, with the structure:

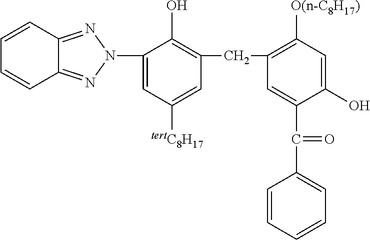

A further preferred class of solid (benzo)triazole UV absorbers is that having the formula:

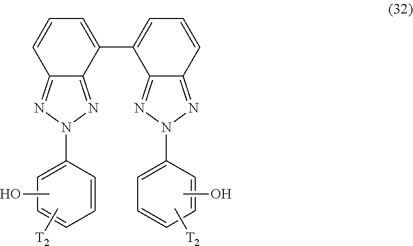

in which T₂ has its previous meaning.

A still further preferred class of solid triazole UV absorbers is that having the formula:

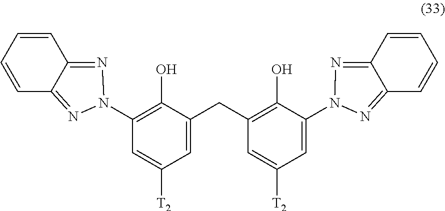

in which T₂ has its previous meaning and is preferably methyl, t-butyl or —C(CH₃)₂—CH₂—C(CH₃)₃. Thus, preferred solid triazole UV absorbers are as follows.

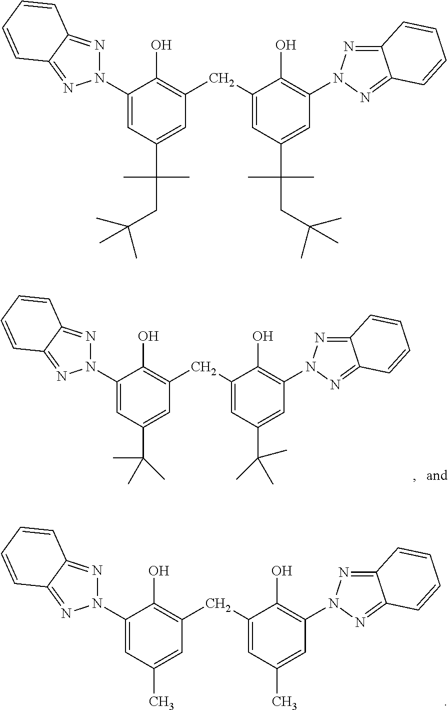 [SMILES: CC(C)(C)CC(C)(C)c1cc(Cc2cc(C(C)(C)CC(C)(C)C)cc(-n3nc4ccccc4n3)c2O)c(O)c(-n2nc3ccccc3n2)c1 ; CC(C)(C)c1cc(Cc2cc(C(C)(C)C)cc(-n3nc4ccccc4n3)c2O)c(O)c(-n2nc3ccccc3n2)c1 ; Cc1cc(Cc2cc(C)cc(-n3nc4ccccc4n3)c2O)c(O)c(-n2nc3ccccc3n2)c1]

More particularly, the particulate organic solid UV filter will be the molecule of formula

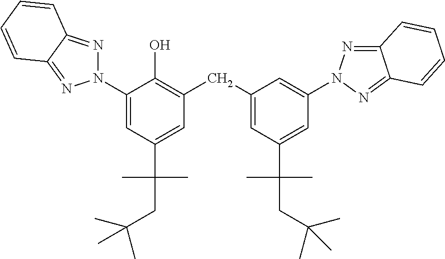 [SMILES: CC(C)(C)CC(C)(C)c1cc(Cc2cc(C(C)(C)CC(C)(C)C)cc(-n3nc4ccccc4n3)c2O)cc(-n2nc3ccccc3n2)c1]

with the nomenclature 2,2′-methylenebis[6-(2H-benzotriazol-2-yl)-4-(1,1,3,3-tetramethylbutyl)phenol], and the INCI Name: METHYLENE BIS-BENZOTRIAZOLYL TETRAMETHYLBUTYLPHENOL as for instance sold under the commercial name TINOSORB M from the company BASF or MIXXIM BB100 from Fairmount Chemical.

A preferred class of solid vinyl group-containing amide UV absorbers is that having the formula:

R₉—(Y)ₘ—CO—C(R₁₀)═C(R₁₁)—N(R₁₂)(R₁₃) (34)

in which R₉ is C₁-C₁₈ alkyl, preferably C₁-C₅ alkyl, or phenyl optionally substituted by one, two or three substituents selected from OH, C₁-C₁₈ alkyl, C₁-C₁₈ alkoxy or CO—OR₆ in which R₆ has its previous meaning; R₁₀, R₁₁, R₁₂ and R₁₃ are the same or different and each is C₁-C₁₈ alkyl, preferably C₁-C₅ alkyl, or hydrogen; Y is N or O; and m has its previous meaning.

Preferred compounds of formula (34) are 4-octyl-3-penten-2-one, ethyl-3-octylamino-2-butenoate, 3-octylamino-1-phenyl-2-buten-1-one and 3-dodecylamino-1-phenyl-2-buten-1-one.

A preferred class of solid cinnamic acid amide UV absorbers is that having the formula:

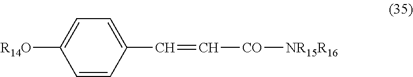

in which R₁₄ is hydroxy or C₁-C₄ alkoxy, preferably methoxy or ethoxy; R₁₅ is hydrogen or C₁-C₄ alkyl, preferably methyl or ethyl; and R₁₆ is —(CONH)ₘ-phenyl in which m has its previous meaning and the phenyl group is optionally substituted by one, two or three substituents selected from OH, C₁-C₁₈ alkyl, C₁-C₁₈ alkoxy or CO—OR₆ in which R₆ has its previous meaning. Preferably R₁₆ is phenyl, 4-methoxyphenyl or the phenylaminocarbonyl group.

Mention may also be made of cinnamamide dimers, such as those disclosed in U.S. Pat. No. 5,888,481, such as, for example, the compound with the structure:

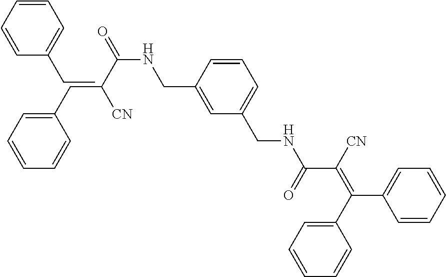

The compounds of formula (1) to (35) are known. The compounds of formula (30) are described, together with their production, in U.S. Pat. No. 4,617,390.

It is preferable that the particulate organic solid UV filter(s) be a benzotriazole derivative, in particular, a phenylbenzotriazole derivative such as a drometrizole trisiloxane, marketed under the trademark “Silatrizole” by Rhodia Chimie or “Mexoryl XL” by L'Oreal, as represented below.

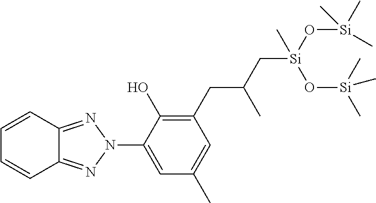 [SMILES: Cc1cc(CC(C)C[Si](C)(O[Si](C)(C)C)O[Si](C)(C)C)c(O)c(-n2nc3ccccc3n2)c1]

Mention may be made, among insoluble organic screening agents of the benzazole type, of those corresponding to one of the following formulae:

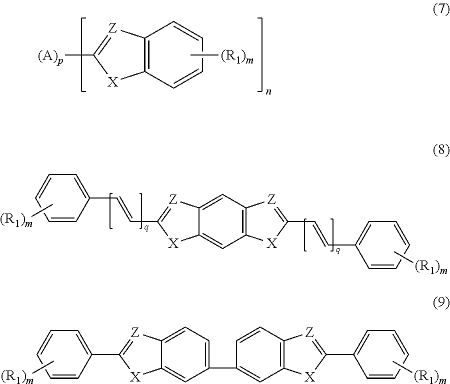

in which each of the X symbols independently represents an oxygen or sulphur atom or an NR₂ group,
each of the Z symbols independently represents a nitrogen atom or a CH group,
each of the R₁ symbols independently represents an OH group, a halogen atom, a linear or branched C₁₋₈ alkyl group, optionally comprising a silicon atom, or a linear or branched C₁₋₈ alkoxy group,
each of the numbers m independently has the value 0, 1 or 2,
n represents an integer between 1 and 4 inclusive,
p is equal to 0 or 1,
each of the numbers q is independently equal to 0 or 1,
each of the R₂ symbols independently represents a hydrogen atom or a benzyl or linear or branched C₁₋₈ alkyl group, optionally comprising a silicon atom,
A represents a radical with a valency n chosen from those of formulae:

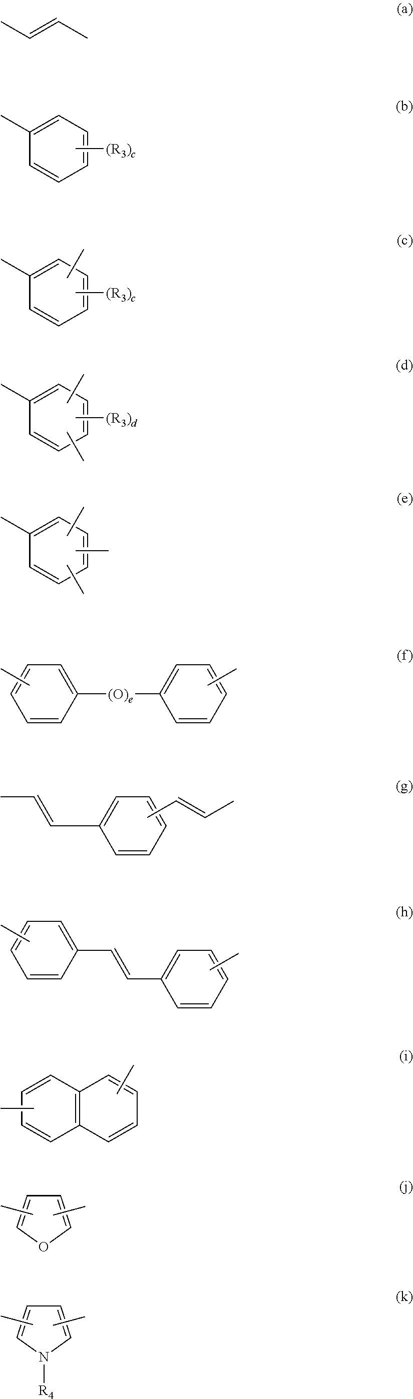

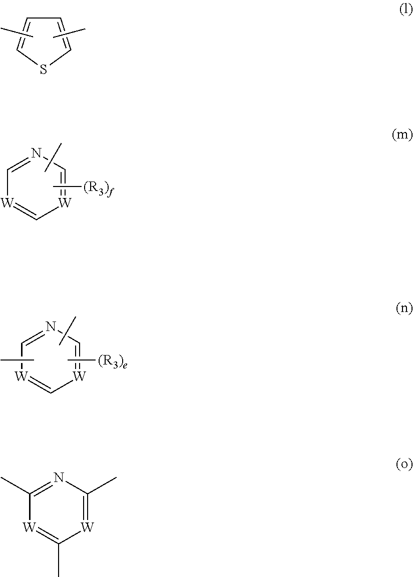

in which W denotes N or CH; each of the R₃ symbols independently represents a halogen atom or a linear or branched C₁₄ alkyl or alkoxy group or a hydroxyl group,
R₄ represents a hydrogen atom or a linear or branched C₁₄ alkyl group, c=0-4, d=0-3, e=0 or 1 and f=0-2.

These compounds are disclosed in particular in Patents DE 676 103 and CH 350 763, U.S. Pat. No. 5,501,850, U.S. Pat. No. 5,961,960, Patent Application EP 0 669 323, U.S. Pat. No. 5,518,713, U.S. Pat. No. 2,463,264, the paper in J. Am. Chem. Soc., 79, 5706-5708, 1957, the paper in J. Am. Chem. Soc., 82, 609-611, 1960, Patent Application EP 0 921 126 and Patent Application EP 712 855.

Mention may be made, as examples of preferred compounds of formula (7) of the family of the 2-arylbenzazoles, of 2-(benzoxazol-2-yl)-4-methylphenol, 2-(1H-benzimidazol-2-yl)-4-methoxyphenol or 2-(benzothiazol-2-yl)phenol, it being possible for these compounds to be prepared, for example, according to the processes disclosed in Patent CH 350 763.

Mention will be made, as examples of preferred compounds of formula (7) of the family of the benzimidazolylbenzazoles, of 2,2′-bisbenzimidazole, 5,5′,6,6′-tetramethyl-2,2′-bisbenzimidazole, 5,5′-dimethyl-2,2′-bisbenzimidazole, 6-methoxy-2,2′-bisbenzimidazole, 2-(1H-benzimidazol-2-yl)benzothiazole, 2-(1H-benzimidazol-2-yl)benzoxazole and N,N′-dimethyl-2,2′-bisbenzimidazole, it being possible for these compounds to be prepared according to the procedures disclosed in U.S. Pat. No. 5,961,960 and U.S. Pat. No. 2,463,264.

Mention will be made, as examples of preferred compounds of formula (7) of the family of the phenylenebenzazoles, of 1,4-phenylenebis(2-benzoxazolyl), 1,4-phenylenebis(2-benzimidazolyl), 1,3-phenylenebis(2-benzoxazolyl), 1,2-phenylenebis(2-benzoxazolyl), 1,2-phenylenebis(benzimidazolyl), 1,4-phenylenebis(N-(2-ethylhexyl)-2-benzimidazolyl) and 1,4-phenylenebis(N-trimethylsilylmethyl-2-benzimidazolyl), it being possible for these compounds to be prepared according to the procedures disclosed in U.S. Pat. No. 2,463,264 and in the publications J. Am. Chem. Soc., 82, 609 (1960) and J. Am. Chem. Soc., 79, 5706-5708 (1957).

Mention will be made, as examples of preferred compounds of formula (7) of the family of the benzofuranylbenzoxazoles, of 2-(2-benzofuranyl)benzoxazole, 2-(benzofuranyl)-5-methylbenzoxazole and 2-(3-methyl-2-benzofuranyl)benzoxazole, it being possible for these compounds to be prepared according to the procedures disclosed in U.S. Pat. No. 5,518,713.

Mention may be made, as preferred compounds of formula (8), of, for example, 2,6-diphenyl-1,7-dihydrobenzo[1,2-d;4,5-d′]diimidazole, corresponding to the formula:

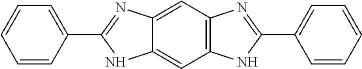 [SMILES: c1ccc(-c2nc3cc4nc(-c5ccccc5)[nH]c4cc3[nH]2)cc1]

or 2,6-distyryl-1,7-dihydrobenzo[1,2-d;4,5-d′]diimidazole or 2,6-di(p-tert-butylstyryl)-1,7-dihydrobenzo[1,2-d;4,5-d′]diimidazole, which compounds can be prepared according to Application EP 0 669 323.

Mention may be made, as preferred compound of formula (9), of 5,5′-bis(2-phenylbenzimidazole) of formula:

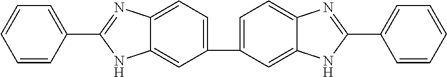 [SMILES: c1ccc(-c2nc3ccc(-c4ccc5nc(-c6ccccc6)[nH]c5c4)cc3[nH]2)cc1]

the preparation of which is described in J. Chim. Phys., 64, 1602 (1967).

Preference is very particularly given, among these solid organic compounds which screen out UV radiation, to 2-(1H-benzimidazol-2-yl)benzoxazole, 6-methoxy-2,2′-bisbenzimidazole,
2-(1H-benzimidazol-2-yl)benzothiazole, 1,4-phenylenebis(2-benzoxazolyl),
1,4-phenylenebis(2-benzimidazolyl), 1,3-phenylenebis(2-benzoxazolyl),
1,2-phenylenebis(2-benzoxazolyl), 1,2-phenylenebis(2-benzimidazolyl) and
1,4-phenylenebis(N-trimethylsilylmethyl-2-benzimidazolyl).

Mention may be made, among solid organic screening agents of the aryl vinylene ketone type, of those corresponding to either of the following formulae (10) and (11):

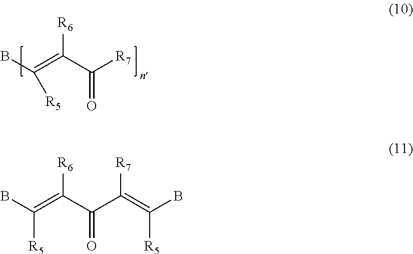

in which:
n′=1 or 2,
   B, in the formula (10) when n′=1 or in the formula (11), is an aryl radical chosen from the following formulae (a′) to (d′) or, in the formula (10) when n′=2, is a radical chosen from the following formulae (e′) to (h′):

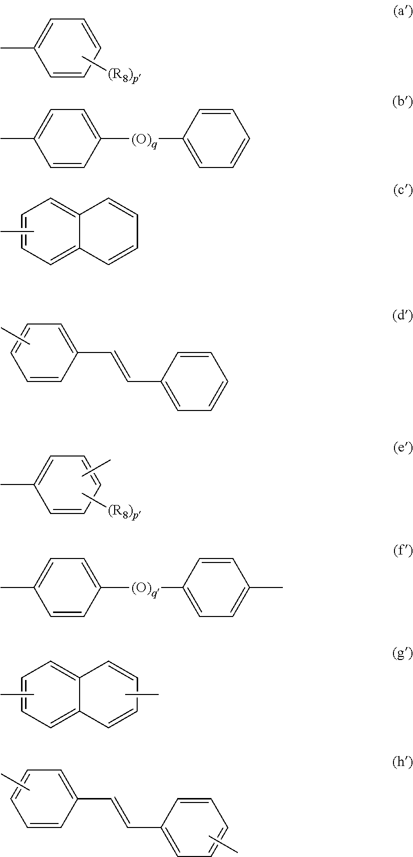

in which:
each of the R₈ symbols independently represents an OH group, a halogen atom, a linear or branched C₁₋₆ alkyl group optionally comprising a silicon atom, a linear or branched C₁₋₆ alkoxy group optionally comprising a silicon atom, a linear or branched C₁₋₅ alkoxycarbonyl group, or a linear or branched C₁₋₆ alkylsulphonamide group optionally comprising a silicon atom or an amino acid functional group,
p′ represents an integer between 0 and 4 inclusive,
q′ represents 0 or 1,
R₅ represents hydrogen or an OH group,
R₆ represents hydrogen, a linear or branched C₁₋₆ alkyl group optionally comprising a silicon atom, a cyano group, a C₁₋₆ alkylsulphonyl group or a phenylsulphonyl group,
R₇ represents a linear or branched C₁₋₆ alkyl group optionally comprising a silicon atom or a phenyl group which can form a bicycle and which is optionally substituted by one or two R₄ radicals,
or R₆ and R₇ together form a monocyclic, bicyclic or tricyclic C₂₋₁₀ hydrocarbonaceous residue, optionally interrupted by one or more nitrogen, sulphur and oxygen atoms and which can comprise another carbonyl, and optionally substituted by a linear or branched C₁-C₅ alkylsulphonamide group, and optionally comprising a silicon atom or an amino acid functional group; provided that, when n′=1, R₆ and R₇ do not form a camphor nucleus.

Mention may be made, as examples of compounds of formula (10) in which n′=1, which screen out UV radiation and which have a mean particle size of between 10 nm and 5 μm, of the following families:
compounds of the styryl ketone type as disclosed in Application JP 04 134 042, such as 1-(3,4-dimethoxyphenyl)-4,4-dimethylpent-1-en-3-one:

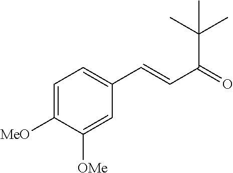 [SMILES: COc1ccc(/C=C/C(=O)C(C)(C)C)cc1OC]

compounds of the benzylidenecineole type, such as those described in the article by E. Mariani et al., 16th IFSCC Congress, New York (1990), for example 1,3,3-trimethyl-5-(4-methoxybenzylidene)-2-oxabicyclo[2.2.2]octan-6-one:

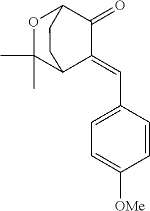 [SMILES: COc1ccc(/C=C2/C(=O)C3CCC2C(C)(C)O3)cc1]

compounds of the benzylidenechromanone type, such as those disclosed in Application JP 04 134 043, for example 3-(4-methoxybenzylidene)-2,3,4a,8a-tetrahydrochromen-4-one:

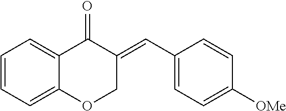 [SMILES: COc1ccc(/C=C2\COc3ccccc3C2=O)cc1]

compounds of the benzylidenethiochromanone type, such as those disclosed in Application JP 04 134 043, for example 3-(4-methoxybenzylidene)-2,3,4a,8a-tetrahydrochromen-4-thione:

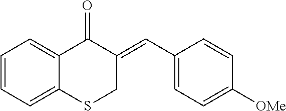 [SMILES: COc1ccc(/C=C2\CSc3ccccc3C2=O)cc1]

compounds of the benzylidenequinuclidinone type, such as those disclosed in Application EP 0 576 974, for example 4-methoxybenzylidene-1-azabicyclo[2.2.2]octan-3-one:

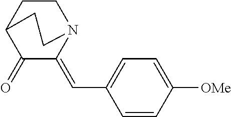 [SMILES: COc1ccc(/C=C2/C(=O)C3CCN2CC3)cc1]

compounds of the benzylidenecycloalkanone type, such as those disclosed in Application FR 2 395 023, for example 2-(4-methoxybenzylidene)cyclopentanone and 2-(4-methoxybenzylidene)cyclohexanone:

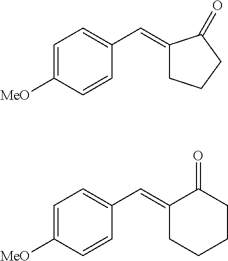 [SMILES: COc1ccc(/C=C2\CCCC2=O)cc1 ; COc1ccc(/C=C2\CCCCC2=O)cc1]

compounds of the benzylidenehydantoin type, such as those disclosed in Application JP 01 158 090, for example 5-(3,4-dimethoxybenzylidene)imidazolidine-2,4-dione:

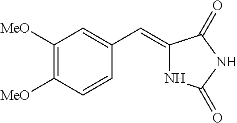 [SMILES: COc1ccc(/C=C2\NC(=O)NC2=O)cc1OC]

compounds of the benzylideneindanone type, such as those disclosed in Application JP 04 134 043, for example 2-(4-methoxybenzylidene)indan-1-one:

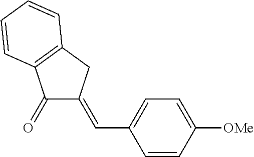 [SMILES: COc1ccc(/C=C2\Cc3ccccc3C2=O)cc1]

compounds of the benzylidenetetralone type, such as those disclosed in Application JP 04 134 043, for example 2-(4-methoxybenzylidene)-3,4-dihydro-2H-naphthalen-1-one;

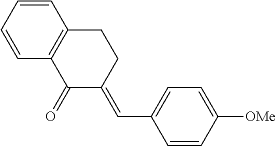 [SMILES: COc1ccc(/C=C2\CCc3ccccc3C2=O)cc1]

compounds of the benzylidenefuranone type, such as those disclosed in Application EP 0 390 683, for example 4-(4-methoxybenzylidene)-2,2,5,5-tetramethyldihydrofuran-3-one:

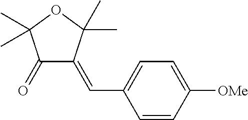 [SMILES: COc1ccc(/C=C2/C(=O)C(C)(C)OC2(C)C)cc1]

compounds of the benzylidenebenzofuranone type, such as those disclosed in Application JP 04 134 041, for example 2-benzylidenebenzofuran-3-one:

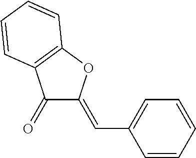 [SMILES: O=C1/C(=C/c2ccccc2)Oc2ccccc21]

compounds of the benzylideneindanedione type, such as 2-(3,5-di(tert-butyl)-4-hydroxybenzylidene)indane-1,3-dione:

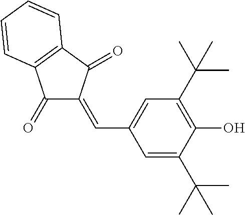 [SMILES: CC(C)(C)c1cc(C=C2C(=O)c3ccccc3C2=O)cc(C(C)(C)C)c1O]

compounds of the benzylidenebenzothiofuranone type, such as those disclosed in Application JP 04,134,043, for example 2-benzylidenebenzo[b]thiophen-3-one:

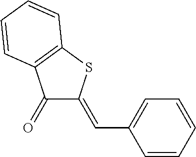 [SMILES: O=C1/C(=C/c2ccccc2)Sc2ccccc21]

compounds of the benzylidenebarbituric type, such as 5-(4-methoxybenzylidene)-1,3-dimethylpyrimidine-2,4,6-trione:

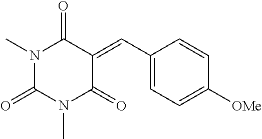 [SMILES: COc1ccc(C=C2C(=O)N(C)C(=O)N(C)C2=O)cc1]

compounds of the benzylidenepyrazolone type, such as 4-(4-methoxybenzylidene)-5-methyl-2-phenyl-2,4-dihydropyrazol-3-one:

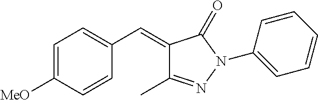 [SMILES: COc1ccc(/C=C2/C(=O)N(c3ccccc3)N=C2C)cc1]

compounds of the benzylideneimidazolone type, such as 5-(4-methoxybenzylidene)-2-phenyl-3,5-dihydroimidazol-4-one:

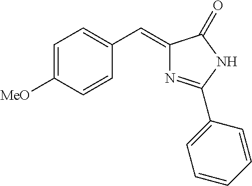 [SMILES: COc1ccc(/C=C2\N=C(c3ccccc3)NC2=O)cc1]

compounds of the chalcone type, such as 1-(2-hydroxy-4-methoxyphenyl)-3-phenylpropenone:

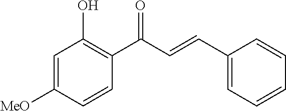 [SMILES: COc1ccc(C(=O)/C=C/c2ccccc2)c(O)c1]

benzylidenone compounds as disclosed in the document FR 2 506 156, for example 3-hydroxy-1-(2-hydroxy-4-methoxyphenyl)-3-phenylpropenone:

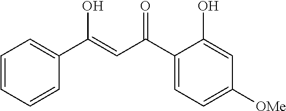 [SMILES: COc1ccc(C(=O)/C=C(\O)c2ccccc2)c(O)c1]

Mention may be made, as examples of compounds of formula (10) in which n′=2, which are insoluble, which screen out UV radiation and which have a mean particle size of between 10 nm and 5 μm, of the following families:
compounds of the phenylenebis(methylidenenorcamphor) type as disclosed in the document EP 0 693 471, for example 1,4-phenylenebis{3-methylidenebicyclo[2.2.1]heptan-2-one}:

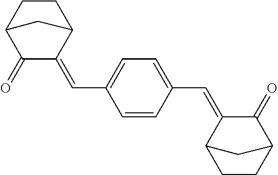 [SMILES: O=C1/C(=C/c2ccc(/C=C3/C(=O)C4CCC3C4)cc2)C2CCC1C2]

compounds of the phenylenebis(methylidenecamphor) type as disclosed in the document FR 2 528 420, for example 1,4-phenylenebis {3-methylidene-1,7,7-trimethylbicyclo[2.2.1]heptan-2-one}:

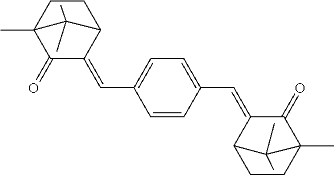

or 1,3-phenylenebis{3-methylidene-1,7,7-trimethylbicyclo[2.2.1]heptan-2-one}:

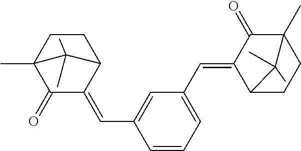

compounds of the phenylenebis(methylidenecamphorsulphonamide) type, such as those disclosed in the document FR 2 529 887, for example 1,4-phenylenebis {3,3′-methylidenecamphor-10,10′-ethylsulphonamide or -(2-ethylhexyl)sulphonamide}:

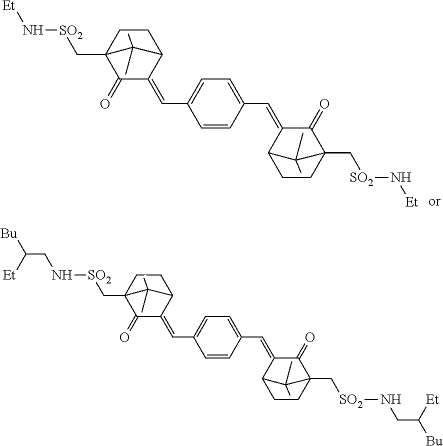

compounds of the phenylenebis(methylidenecineole) type as described in the paper by E. Mariani et al., 16th IFSCC Congress, New York (1990), for example 1,4-phenylenebis {5-methylidene-3,3-dimethyl-2-oxabicyclo[2.2.2]octan-6-one}:

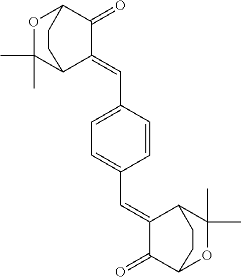

compounds of the phenylenebis(methylideneketotricyclodecane) type as disclosed in Application EP 0 694 521, such as 1,4-phenylenebis(octahydro-4,7-methano-6-inden-5-one):

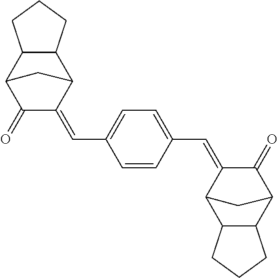 [SMILES: O=C1/C(=C/c2ccc(/C=C3/C(=O)C4CC3C3CCCC43)cc2)C2CC1C1CCCC21]

compounds of the phenylenebis(alkylene ketone) type, such as those disclosed in Application JP 04 134 041, for example 1,4-phenylenebis(4,4-dimethylpent-1-en-3-one):

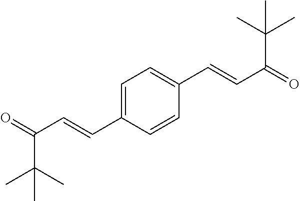 [SMILES: CC(C)(C)C(=O)/C=C/c1ccc(/C=C/C(=O)C(C)(C)C)cc1]

compounds of the phenylenebis(methylidenefuranone) type as disclosed in Application FR 2 638 354, for example 1,4-phenylenebis(4-methylidene-2,2,5,5-tetramethyldihydrofuran-3-one):

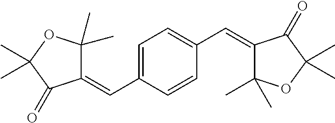

compounds of the phenylenebis(methylidenequinuclidinone) type, such as those disclosed in Application EP 0 714 880, for example 1,4-phenylenebis{2-methylidene-1-azabicyclo[2.2.2]octan-3-one}:

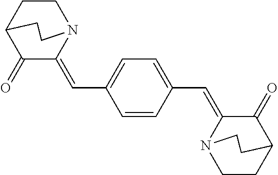 [SMILES: O=C1/C(=C/c2ccc(/C=C3/C(=O)C4CCN3CC4)cc2)N2CCC1CC2]

Mention may be made, as compounds of formula (11), of the following families:
compounds of the bis(benzylidene)cycloalkanone type, such as 2,5-di(benzylidene)cyclopentanone:

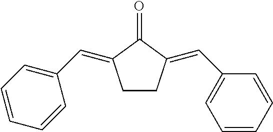 [SMILES: O=C1/C(=C/c2ccccc2)CC/C1=C\c1ccccc1]

compounds of the γ-pyrone type as disclosed in the document JP 04 290 882, for example 2,6-bis(3,4-dimethoxyphenyl)pyran-4-one:

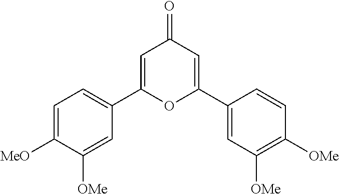 [SMILES: COc1ccc(-c2cc(=O)cc(-c3ccc(OC)c(OC)c3)o2)cc1OC]

Preference is very particularly given, among these insoluble organic compounds which screen out UV radiation of the aryl vinylene ketone type, to the compounds of formula (10) in which n′=2.

Mention may be made, among solid organic screening agents of the phenylenebis(benzoxazinone) type, of those corresponding to the following formula (12):

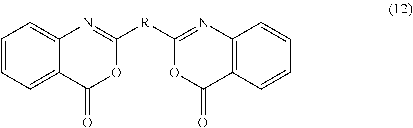

with R representing a divalent aromatic residue chosen from the following formulae (e″) to (h″):

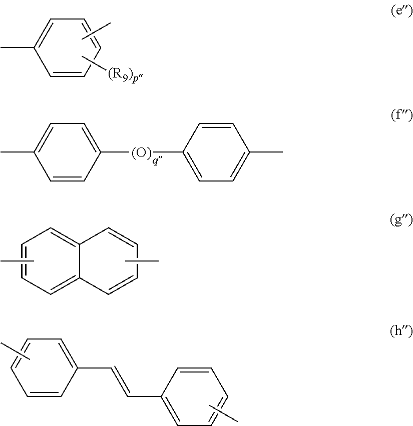

in which:
each of the R₉ symbols independently represents an OH group, a halogen atom, a linear or branched C₁₋₆ alkyl group optionally comprising a silicon atom, a linear or branched C₁₋₆ alkoxy group optionally comprising a silicon atom, a linear or branched C₁₋₅ alkoxycarbonyl group, or a linear or branched C₁₋₆ alkylsulphonamide group optionally comprising a silicon atom or an amino acid functional group,
p″ represents an integer between 0 and 4 inclusive, and
q″ represents 0 or 1.

Mention may be made, as examples of compounds of formula (12), which are insoluble, which screen out UV radiation and which have a mean particle size of between 10 nm and 5 μm, of the following derivatives:
2,2′-p-phenylenebis(3,1-benzoxazin-4-one), commercial product Cyasorb UV-3638 from Cytec,
2,2′-(4,4′-biphenylene)bis(3,1-benzoxazin-4-one),
2,2′-(2,6-naphthylene)bis(3,1-benzoxazin-4-one).

Mention may be made, among solid organic screening agents of the acrylonitrile amide, sulphonamide or carbamate derivative type, of those corresponding to the following formula (13):

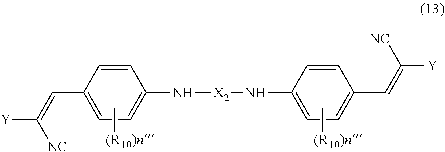

in which:
R₁₀ represents a linear or branched C₁₋₈ alkyl group,
n has the value 0, 1 or 2,
X₂ represents a divalent radical of formula —(C═O)—R₁₁—(C═O)—, —SO₂—R₁₁—SO₂— or —(C═O)—O—R₁₁—O—(C═O)—,
Y represents a —(C═O)—R₁₂ or —SO₂R₁₃ radical,
R₁₁ represents a single bond or a linear or branched, divalent C₁-C₃₀ alkylene or C₃-C₃₀ alkenylene radical which can carry one or more hydroxyl substituents and which can comprise, in the carbonaceous chain, one or more heteroatoms chosen from oxygen, nitrogen and silicon atoms,
R₁₂ represents an —OR₁₄ or —NHR₁₄ radical,
R₁₃ represents a linear or branched C₁-C₃₀ alkyl radical or a phenyl ring which is unsubstituted or substituted by C₁-C₄ alkyl or alkoxy radicals, and
R₁₄ represents a linear or branched C₁-C₃₀ alkyl or C₃-C₃₀ alkenyl radical which can carry one or more hydroxyl substituents and which can comprise, in the carbonaceous chain, one or more heteroatoms chosen from oxygen, nitrogen and silicon atoms.

Although only the isomers in which the cyano substituent is in the cis position with respect to the para-aminophenyl substituent are represented in the above formula (13), this formula should be understood as also encompassing the corresponding trans isomers; for each of the two double bonds, and independently, the cyano and para-aminophenyl substituents can be in the cis or trans configuration with respect to one another.

Mention may be made, as example, of the dimer of 2-ethylhexyl 2-cyano-3-[4-(acetylamino)phenyl]acrylate of formula:

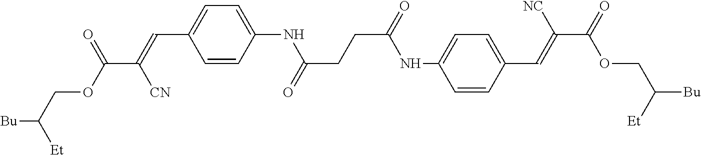

Another specific family of solid organic screening agents in accordance with the invention are the polyvalent metal salts (for example, Ca²⁺, Zn²⁺, Mg²⁺, Ba²⁺, Al³⁺ or Zr⁴⁺) of sulphonic or carboxylic organic screening agents, such as the polyvalent metal salts of sulphonated derivatives of benzylidenecamphor, such as those disclosed in Application FR-A 2 639 347; the polyvalent metal salts of sulphonated derivatives of benzimidazole, such as those disclosed in Application EP-A-893 119; or the polyvalent metal salts of cinnamic acid derivatives, such as those disclosed in Application JP-87 166 517.

Mention may also be made of metal or ammonium or substituted ammonium complexes of UV-A and/or UVB organic screening agents as disclosed in Patent Applications WO 93/10753, WO 93/11095 and WO 95/05150.

A preferred class of particulate solid sulfonated benzimidazole UV absorbers is that having the formula:

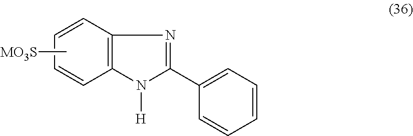

in which M is hydrogen or an alkali metal, preferably sodium, an alkaline earth metal, such as magnesium or calcium, or zinc.

In the compounds of formula (1) to (35), C₁-C₁₈ alkyl groups may be methyl, ethyl, n-propyl, isopropyl, n-butyl, isobutyl, tert-butyl, n-amyl, n-hexyl, n-heptyl, n-octyl, iso-octyl, n-nonyl, n-decyl, n-undecyl, n-dodecyl, tetradecyl, hexydecyl or octadecyl; and C₁-C₁₈ alkoxy groups include methoxy, ethoxy, propoxy, butoxy, n-hexoxy, n-heptoxy, n-octoxy, iso-octoxy, n-nonoxy, n-decoxy, n-undecoxy, n-dodecoxy, tetradecoxy, hexadecoxy or octadecoxy, methoxy and ethoxy being preferred.

C₁-C₁₈ carboxyalkyl includes carboxymethyl, carboxyethyl, carboxypropyl, carboxyisopropyl, carboxybutyl, carboxyisobutyl, carboxybutyl, carboxyamyl, carboxyhexyl, carboxyheptyl, carboxyoctyl, carboxyisooctyl, carboxynonyl, carboxydecyl, carboxyundecyl, carboxydodecyl, carboxytetradecyl, carboxyhexadecyl and carboxyoctadecyl, carboxymethyl being preferred.

C₅-C₈ cycloalkyl includes cyclopentyl, cyclohexyl and cyclooctyl.

The particulate organic solid UV filter may be selected from the group consisting of benzotriazole derivatives, oxanilide derivatives, triazine derivatives, triazole derivatives, vinyl-group containing amides, cinnamic acid amides, and sulfonated benzimidazoles.

It is also preferable that the particulate organic solid UV filter(s) is selected from methylenebis(hydroxyphenylbenzotriazole) derivatives in the form of a solid.

Methylene bis-benzotriazolyl tetramethylbutylphenol, such as 2,2′-methylenebis[6-(2H-benzotriazol-2-yl)-4-methyl-phenol] marketed in the solid form under the trademark “Mixxim BB/200” by Fairmount Chemical, or 2,2′-methylenebis[6-(2H-benzotriazol-2-yl)-4-(1,1,3,3-tetramethylbutyl)phenol] marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071, 5,166,355, GB-2,303,549, DE-197,26,184 and EP-893,119, are in particular preferable.

The particulate organic solid UV filter(s) may be used in the composite pigment in proportions such that the weight ratio of the small core particle to the particulate organic solid UV filter(s) is from 10:90 to 90:10, preferably from 30:70 to 80:20, and more preferably from 40:60 to 50:50.

### (Coloring Pigment)

As described above, the coating layer(s) on the small core particle may comprise at least one coloring pigment.

The term “coloring pigment(s)” should be understood as meaning white or colored, inorganic or organic particle(s) of any shape which is/are insoluble and is/are intended to color a composition comprising them.

If coloring pigment(s) is/are used, the composite pigment has an effect of providing a clearer appearance with high chroma, because the coloring pigments do not aggregate but spread on the substrate. It should be noted that free coloring pigments easily aggregate to give a dark appearance with low chroma to the skin. Therefore, the color of the cosmetics including coloring pigments can be opaque and dark. On the other hand, the composite pigment according to the present invention can provide clear and bright color tone.

The pigments can be white or colored, inorganic and/or organic and generally have a mean particle size greater or equal to 1 μm.

Among the inorganic pigments that may be used, non-limiting mention may be made of titanium dioxide, optionally surface treated, zirconium or cerium oxide, as well as zinc, (black, yellow or red) iron or chromium oxide, manganese violet, ultramarine blue, chromium hydrate and ferric blue, barium sulfate, or metal powders, such as aluminum, copper, silver or gold powder.

The particle size of the coloring pigment is not limited. In a particular embodiment, the coloring pigment may have a mean particle size of from 100 nm to less than 1 μm, preferably from 100 nm to less than 500 nm, and more preferably from 100 nm to less than 300 nm.

Since particles of coloring pigment(s) can be firmly bonded on the small core particle, the coloring pigment(s) cannot penetrate into the skin via pores on the skin. In addition, even if the coloring pigment(s) irritate, a large amount of the coloring pigment(s) cannot directly contact with the skin, because they are present only on the small core particle. Accordingly, the composite pigment according to the present invention is safer than the bulk of coloring pigments.

Among organic pigments that may be used, non-limiting mention may be made of carbon black, pigments of D&C type and lakes, such as lakes-based on cochineal carmine and on barium, strontium, calcium or aluminum. For example, Red 202 (Calcium bis[2-(3-carboxy-2-hydroxynephthylazo)-5-methylbenzenesulfonate) may be used as the pigment of D&C type.

Preferably, the coloring pigment is chosen from titanium dioxide, zirconium oxide, cerium oxide, zinc oxide, iron oxide, chromium oxide, manganese violet, ultramarine blue, chromium hydrate, ferric blue, aluminum powder, copper powder, silver powder, gold powder, barium sulfate, carbon black, pigments of D&C type, lakes, pearlescent pigments, and mixtures thereof.

The term “pearlescent pigments” should be understood as meaning iridescent particles of any shape, such as particles produced by certain shellfish in their shells or else synthesized.

The pearlescent agents can be chosen from white pearlescent agents, such as mica covered with titanium dioxide or with bismuth oxychloride; colored pearlescent agents, such as titanium oxide-coated mica covered with iron oxide, titanium oxide-coated mica covered with ferric blue or chromium oxide, or titanium oxide-coated mica covered with an organic pigment of the abovementioned type; and pearlescent agents based on bismuth oxychloride.

The composite pigment used in the present invention can provide a better feeling on use, because fine particles of coloring pigment(s), if used, can be firmly fixed on the small core particles so that it is possible to reduce free fine particles which have a high friction coefficient such that they do not easily spread on the skin and provide an unpleasant feeling on use.

The coloring pigment(s) may be used in the composite pigment in proportions such that the weight ratio of the small core particle to the coloring pigment(s) is from 50:50 to 90:10, preferably from 50:50 to 80:20, and more preferably from 50:50 to 70:30.

### (Additional UV Filter)

As described above, the coating layer on the small core particle may further comprise at least one additional UV filter. If two or more additional UV filters are used, they may be the same or different, preferably the same.

The additional UV filter used for the present invention may be active in the UV-A and/or UV-B region, preferably in the UV-A region or in the UV-A and UVB region. The additional UV filter may be hydrophilic and/or lipophilic.

The additional UV filter may be solid or liquid, preferably liquid. The terms “solid” and “liquid” mean solid and liquid, respectively, at 25° C. under 1 atm. The additional UV filter may be made from at least one organic or inorganic material, preferably at least one organic material.

The additional LTV filter(s) may be selected from the group consisting of anthranilic derivatives; dibenzoylmethane derivatives; cinnamic derivatives; salicylic derivatives; camphor derivatives; benzophenone derivatives; β,β-diphenylacrylate derivatives; triazine derivatives; benzotriazole derivatives; benzalmalonate derivatives; benzimidazole derivatives; imidazoline derivatives; bis-benzoazolyl derivatives; p-aminobenzoic acid (PABA) and derivatives thereof; methylenebis(hydroxyphenylbenzotriazole) derivatives; benzoxazole derivatives; screening polymers and screening silicones; dimers derived from α-alkylstyrene; 4,4-diarylbutadienes; octocrylene and derivatives thereof, guaiazulene and derivatives thereof, rutin and derivatives thereof, flavonoids, biflavonoids, oryzanol and derivatives thereof, quinic acid and derivatives thereof, phenols, retinol, cysteine, aromatic amino acids, peptides having an aromatic amino acid residue, and mixtures thereof.

Mention may be made, as examples of the additional organic UV filter(s), of those denoted below under their INCI names, and mixtures thereof.
Anthranilic derivatives: Menthyl anthranilate, marketed under the trademark “Neo Heliopan MA” by Haarmann and Reimer.
Dibenzoylmethane derivatives: Butyl methoxydibenzoylmethane, marketed in particular under the trademark “Parsol 1789” by Hoffmann-La Roche; and isopropyl dibenzoylmethane.
Cinnamic derivatives: Ethylhexyl methoxycinnamate, marketed in particular under the trademark “Parsol MCX” by Hoffmann-La Roche; isopropyl methoxycinnamate; isopropoxy methoxycinnamate; isoamyl methoxycinnamate, marketed under the trademark “Neo Heliopan E 1000” by Haarmann and Reimer; cinoxate (2-ethoxyethyl-4-methoxy cinnamate); DEA methoxycinnamate; diisopropyl methylcinnamate; and glyceryl ethylhexanoate dimethoxycinnamate.
Salicylic derivatives: Homosalate (homomentyl salicylate), marketed under the trademark “Eusolex HMS” by Rona/EM Industries; ethylhexyl salicylate, marketed under the trademark “Neo Heliopan OS” by Haarmann and Reimer; glycol salicylate; butyloctyl salicylate; phenyl salicylate; dipropyleneglycol salicylate, marketed under the trademark “Dipsal” by Scher; and TEA salicylate, marketed under the trademark “Neo Heliopan TS” by Haarmann and Reimer.
Camphor derivatives, in particular, benzylidenecamphor derivatives: 3-benzylidene camphor, manufactured under the trademark “Mexoryl SD” by Chimex; 4-methylbenzylidene camphor, marketed under the trademark “Eusolex 6300” by Merck; benzylidene camphor sulfonic acid, manufactured under the trademark “Mexoryl SL” by Chimex; camphor benzalkonium methosulfate, manufactured under the trademark “Mexoryl SO” by Chimex; terephthalylidene dicamphor sulfonic acid, manufactured under the trademark “Mexoryl SX” by Chimex; and polyacrylamidomethyl benzylidene camphor, manufactured under the trademark “Mexoryl SW” by Chimex.
Benzophenone derivatives: Benzophenone-1 (2,4-dihydroxybenzophenone), marketed under the trademark “Uvinul 400” by BASF; benzophenone-2 (Tetrahydroxybenzophenone), marketed under the trademark “Uvinul D50” by BASF; Benzophenone-3 (2-hydroxy-4-methoxybenzophenone) or oxybenzone, marketed under the trademark “Uvinul M40” by BASF; benzophenone-4 (hydroxymethoxy benzophonene sulfonic acid), marketed under the trademark “Uvinul MS40” by BASF; benzophenone-5 (Sodium hydroxymethoxy benzophenone Sulfonate); benzophenone-6 (dihydroxy dimethoxy benzophenone); marketed under the trademark “Helisorb 11” by Norquay; benzophenone-8, marketed under the trademark “Spectra-Sorb UV-24” by American Cyanamid; benzophenone-9 (Disodium dihydroxy dimethoxy benzophenonedisulfonate), marketed under the trademark “Uvinul DS-49” by BASF; benzophenone-12, and n-hexyl 2-(4-diethylamino-2-hydroxybenzoyl)benzoate.
β,β-Diphenylacrylate derivatives: Octocrylene, marketed in particular under the trademark “Uvinul N539” by BASF; and Etocrylene, marketed in particular under the trademark “Uvinul N35” by BASE
Triazine derivatives: diethylhexyl butamido triazone, marketed under the trademark “Uvasorb HEB” by Sigma 3V; 2,4,6-tris(dineopentyl 4′-aminobenzalmalonate)-s-triazine.
Benzotriazole derivatives, in particular, phenylbenzotriazole derivatives: 2-(2H-benzotriazole-2-yl)-6-dodecyl-4-methylpheno, branched and linear; and those described in U.S. Pat. No. 5,240,975.
Benzalmalonate derivatives: Dineopentyl 4′-methoxybenzalmalonate, and polyorganosiloxane comprising benzalmalonate functional groups, such as polysilicone-15, marketed under the trademark “Parsol SLX” by Hoffmann-LaRoche.
Benzimidazole derivatives, in particular, phenylbenzimidazole derivatives: Phenylbenzimidazole sulfonic acid, marketed in particular under the trademark “Eusolex 232” by Merck, and disodium phenyl dibenzimidazole tetrasulfonate, marketed under the trademark “Neo Heliopan AP” by Haarmann and Reimer.
Imidazoline derivatives: Ethylhexyl dimethoxybenzylidene dioxoimidazoline propionate.
Bis-benzoazolyl derivatives: The derivatives as described in EP-669,323 and U.S. Pat. No. 2,463,264.
Para-aminobenzoic acid and derivatives thereof: PABA (p-aminobenzoic acid), ethyl PABA, Ethyl dihydroxypropyl PABA, pentyl dimethyl PABA, ethylhexyl dimethyl PABA, marketed in particular under the trademark “Escalol 507” by ISP, glyceryl PABA, and PEG-25 PABA, marketed under the trademark “Uvinul P25” by BASF.
Benzoxazole derivatives:
2,4-bis[5-1(dimethylpropyl)benzoxazol-2-yl-(4-phenyl)imino]-6-(2-ethylhexyl)imino-1,3,5-triazine, marketed under the trademark of Uvasorb K2A by Sigma 3V.
Screening polymers and screening silicones: The silicones described in WO 93/04665.
Dimers derived from α-alkylstyrene: The dimers described in DE-19855649.
4,4-Diarylbutadiene derivatives: 1,1-dicarboxy(2,2′-dimethylpropyl)-4,4-diphenylbutadiene.
Quaiazulene and derivatives thereof: Guaiazulene and sodium guaiazulene sulfonate.
Rutin and derivatives thereof: Rutin and glucosylrutin.
Flavonoids: Robustin (isoflavonoid), genistein (flavonoid), tectochrysin (flavonoid), and hispidone (flavonoid).
Biflavonoids: Lanceolatin A, lanceolatin B, and hypnumbiflavonoid A.
Oryzanol and derivatives thereof: F-oryzanol.
Quinic acid and derivatives thereof: Quinic acid.
Phenols: Phenol.
Retinols: Retinol.
Cysteines: L-cysteine.
Peptides having an aromatic amino acid residue: Peptides having tryptophan, tyrosine or phenylalanine.

The preferred organic additional UV filter(s) is selected from:

butyl methoxydibenzoylmethane, ethylhexyl methoxycinnamate, homosalate, ethylhexyl salicylate, octocrylene, phenylbenzimidazole sulfonic acid, benzophenone-3, benzophenone-4, benzophenone-5, n-hexyl 2-(4-diethylamino-2-hydroxybenzoyl)benzoate, 4-methylbenzylidene camphor, terephthalylidene dicamphor sulfonic acid, disodium phenyl dibenzimidazole tetrasulfonate, ethylhexyl triazone, bis-ethylhexyloxyphenol methoxyphenyl triazine, diethylhexyl butamido triazone, 2,4,6-tris(dineopentyl 4′-aminobenzalmalonate)-s-triazine, 2,4,6-tris(diisobutyl 4′-aminobenzalmalonate)-s-triazine, methylene bis-benzotriazolyl tetramethylbutylphenol, polysilicone-15, dineopentyl 4′-methoxybenzalmalonate, 1,1-dicarboxy(2,2′-dimethylpropyl)-4,4-diphenylbutadiene, 2,4-bis[5-1(dimethylpropyl)benzoxazol-2-yl-(4-phenyl)imino]-6-(2-ethylhexyl)imino-1,3,5-triazin e, and their mixtures. A more preferable organic UV filter is butyl methoxydibenzoylmethane (Avobenzone).

In a preferred embodiment, the additional UV filter is an organic liquid UV filter.

The material of the organic liquid UV filter(s) is not limited as long as it is organic. If two or more organic liquid UV filters are used, the material(s) of the organic liquid UV filters may be the same as or different from each other.

Amongst the liquid additional organic UV filter, we can mention:
Cinnamic derivatives: Ethylhexyl methoxycinnamate, marketed in particular under the trademark “Parsol MCX” by Hoffmann-La Roche; isopropyl methoxycinnamate; isopropoxy methoxycinnamate; isoamyl methoxycinnamate, marketed under the trademark “Neo Heliopan E 1000” by Haarmann and Reimer; cinoxate (2-ethoxyethyl-4-methoxy cinnamate); DEA methoxycinnamate; diisopropyl methylcinnamate; and glyceryl ethylhexanoate dimethoxycinnamate.
Salicylic derivatives: Homosalate (homomentyl salicylate), marketed under the trademark “Eusolex HMS” by Rona/EM Industries; ethylhexyl salicylate, marketed under the trademark “Neo Heliopan OS” by Haarmann and Reimer; glycol salicylate; butyloctyl salicylate; phenyl salicylate; dipropyleneglycol salicylate, marketed under the trademark “Dipsal” by Scher; and TEA salicylate, marketed under the trademark “Neo Heliopan TS” by Haarmann and Reimer.
β,β-Diphenylacrylate derivatives: Octocrylene, marketed in particular under the trademark “Uvinul N539” by BASF; and Etocrylene, marketed in particular under the trademark “Uvinul N35” by BASF.
Polyorganosiloxane comprising benzalmalonate functional groups, such as polysilicone-15, marketed under the trademark “Parsol SLX” by Hoffmann-LaRoche.

The preferred organic liquid additional UV filter(s) may be selected from:

ethylhexyl methoxycinnamate, homosalate, ethylhexyl salicylate, octocrylene, polysilicone-15.

The additional UV filter(s) may be used in the composite pigment in proportions such that the weight ratio of the small core particle to the additional UV filter(s) is from 50:50 to 90:10, preferably from 50:50 to 80:20, and more preferably from 50:50 to 70:30.

### (Large Core Particle)

The composite pigment used in the present invention may further comprise at least one large core particle. It is preferable that the composite pigment comprise at least one large core particle.

The large core particle to be used for the composite pigment is not limited, as long as the large core particle has a mean particle size or a mean particle diameter of 2 μm or more, preferably 3 μm or more, more preferably 4 μm or more, and even more preferably 5 μm or more. The mean particle size of the large core particle may be limited to 50 μm or less, preferably 30 μm or less, and more preferably 20 μm or less, and even more preferably 10 μm or less.

The mean particle size or mean particle diameter here is an arithmetic mean diameter, and can be determined, for example, by calculating the average of the dimensions of one hundred particles chosen on an image obtained with a scanning electron microscope.

The large core particle may be hollow or solid. It may be preferable to use solid large particle.

The large particle can be in any shape. For example, it is possible to use a large particle in the form of a plate with an aspect ratio of at least 5, preferably more than 10, more preferably more than 20, and more preferably more than 50. The aspect ratio can be determined by the average thickness and the average length according to the formula: aspect ratio=length/thickness.

If a plate-like particle is used for the present invention, it is preferable that the plate-like particle have a length ranging from 2 μm or more, preferably 3 μm or more, more preferably 4 pin or more, and even more preferably 5 μm or more, to 50 μm or less, preferably 30 μm or less, and more preferably 20 μm or less, and even more preferably 10 μm or less.

In a preferred embodiment, the large core particle has a spherical shape.

The material of the large core particle is not limited. The material can be at least one inorganic material and/or at least one organic material, preferably at least one organic material.

The inorganic material and/or organic material may be hollow or porous. The porosity of the material may be characterized by a specific surface area of from 0.05 m²/g to 1,500 m²/g, more preferably from 0.1 m²/g to 1,000 m²/g, and more preferably from 0.2 m²/g to 500 m²/g according to the BET method.

Preferably, the inorganic material can be selected from the group consisting of mica, synthetic mica, talc, sericite, boron nitride, glass flakes, calcium carbonate, barium sulfate, titanium oxide, hydroxyapatite, silica, silicate, zinc oxide, magnesium sulfate, magnesium carbonate, magnesium trisilicate, aluminum oxide, aluminum silicate, calcium silicate, calcium phosphate, magnesium oxide, bismuth oxychloride, kaolin, hydrotalcite, mineral clay, synthetic clay, iron oxide, and mixtures thereof. Natural mica, synthetic mica, sericite, kaolin, talc, silica and mixtures thereof are more preferable.

In particular, silica particles such as P-1500 marketed by JGC C&C are preferable as inorganic large particles.

Preferably, the organic material can be selected from the group consisting of poly(meth)acrylates, polyamides, silicones, polyurethanes, polyethylenes, polypropylenes, polystyrenes, copolystyrenes, polyhydroxyalkanoates, polycaprolactams, poly(butylene) succinates, polysaccharides, polypeptides, polyvinyl alcohols, polyvinyl resins, fluoropolymers, waxes, amidosulfonic acid polyvalent metal salts, acylated amino acids, and mixtures thereof. As the fluoropolymers, for example, PTFE may be used. As the amidosulfonic acid polyvalent metal salts, for example, N-lauroyltaurine calcium may be used. As the acylated amino acids, lauroyllysine may be used. Polyamides such as Nylon®, polyhydroxyalkanoates such as polylactic acids, poly(meth)acrylates such as polymethylmethacrylates, silicones, fluoropolymers, and mixtures thereof are more preferable.

In particular, polymethylmethacrylate particles such as MR-7GC marketed by Soken in Japan, polyamide particles such as SP-500 marketed by Toray, Orgasol marketed by Arkema, and PTFE particles such as Ceridust 9205F marketed by Clariant, are preferable as organic large core particles.

The large core particle may or may not be coated beforehand. In a particular embodiment, the large core particle is originally coated. The material of an original coating of the large core particle is not limited, but an organic material such as an amino acid, an N-acylamino acid, an amido, a silicone, a modified silicone and a polyolefin, is preferable. As the organic material, mention may be made of lauroyl lysine, acryl-modified silicone and polyethylene.

In particular, silica particles coated with polyethylene such as ACEMATT OK412 marketed by Degussa may be preferable as coated (inorganic) large particles.

In the composite pigment used in the present invention, the weight ratio of the small core particle(s) to the large core particle(s) may be from 10:90 to 90:10, preferably from 20:80 to 80:20, and more preferably from 30:70 to 70:30.

In a particular embodiment, the weight ratio of the small core particle(s)/the large core particle(s)/the inorganic or organic particulate solid UV filter(s) may be from 20:50:30 to 50:20:30, preferably from 35:15:50 to 15:35:50, and more preferably from 10:20:70 to 20:10:70.

In a preferred embodiment, the weight ratio of the small core particle(s)/the large core particle(s)/the inorganic or organic particulate solid UV filter(s) may be from 50:20:30 or 35:15:50.

In a preferred embodiment, the composite pigment may satisfy the following requirements: the small particle comprises at least one copolystyrene, preferably a styrene/acrylate copolymer, and/or a cross-linked styrene/methyl methacrylate copolymer;

the large particle comprises at least one poly(meth)acrylate, preferably a methyl methacrylate polymer; and
the small and large particles are at least in part covered with at least one coating layer comprising an inorganic solid UV filter selected from metal oxide such as titanium oxide.

### (Method for Preparing Composite Pigment)

One embodiment of the composite pigments used in the present invention can be prepared by subjecting

at least one small particle with a mean particle size more than 100 nm and of less than 1 μm, preferably less than 600 nm, and more preferably less than 400 nm;
at least one inorganic or organic particulate solid UV filter; and
optionally at least one coloring pigment and/or at least one additional UV filter to a mechanochemical fusion process.

Another embodiment of the composite pigment used in the present invention can be prepared by can be obtained by subjecting:

at least one small particle with a mean particle size more than 100 nm and of less than 1 μm, preferably less than 600 nm, and more preferably less than 400 nm;
at least one large particle with a mean particle size of 2 μm or more, preferably 3 μm or more, more preferably 4 μm or more, and even more preferably 5 μm or more;
at least one inorganic or organic particulate solid UV filter; and
optionally at least one coloring pigment and/or at least one additional UV filter to a mechanochemical fusion process.

The small core particle, the large core particle, the inorganic or organic particulate solid UV filter, the coloring pigment, and the additional UV filter are as explained above.

Mechanochemical fusion process means a process in which mechanical power such as impact force, friction force or shear force is applied to a plurality of subjects to cause fusion between the subjects.

The mechanochemical fusion process may be performed by, for example, an apparatus comprising a rotating chamber and a fixed inner piece with a scraper, such as a mechanofusion system marketed by Hosokawa Micron Corporation in Japan.

It is preferable to use a hybridizer process as the mechanochemical fusion process.

The hybridizer process was developed in the 1980s. The hybridizer process is a class of mechanochemical fusion processes in which strong mechanical power is applied to a plurality of particles to cause a mechanochemical reaction to form a composite particle.

According to the hybridizer process, the mechanical power is imparted by a high-speed rotor which can have a diameter from 10 cm to 1 m, and can rotate at a speed of 1,000 rpm to 100,000 rpm. Therefore, the hybridizer process can be defined as a mechanochemical fusion process using such a high-speed rotor. The hybridizer process is performed in air or under dry conditions. Thus, due to the high-speed rotation of the rotor, high-speed air flow may be generated near the rotor. However, some liquid materials may be subjected to the hybridizer process together with solid materials. The term “hybridizer process” has been used as a technical term.

The hybridizer process can be performed by using a hybridization system marketed by, for example, Nara Machinery in Japan, in which at least two types of particles, typically core particles and fine particles, are fed into a hybridizer equipped with a high-speed rotor having a plurality of blades in a chamber under dry conditions, and the particles are dispersed in the chamber and mechanical and thermal energy (e.g., compression, friction and shear stress) are imparted to the particles for a relatively short period of time such as 1 to 10 minutes, preferably 1 to 5 minutes.

As a result, one type of particles (e.g., fine particles) is embedded or fixed on the other type of particles (e.g., core particle) to form composite particles. It is preferable that the particles have been subjected to electrostatic treatment(s) such as shaking to form an “ordered mixture” in which one type of particles is spread to cover the other type of particles. The hybridizer process can also be performed by using a theta composer marketed by Tokuju Corporation in Japan.

The hybridizer process can also be performed by using a Composi Hybrid or a Mechano Hybrid marketed by Nippon coke.

According to the present invention, for example, small core particles and inorganic or organic particulate solid UV filter(s) as well as optionally additional material(s) such as large core particles, coloring pigment(s) and/or additional UV filter(s) if necessary, can be fed into such a hybridizer to form a composite pigment. The hybridizer process can be performed by using a rotor rotating at about 8,000 rpm (100 msec) for about 3 minutes.

If the large core particles are used, the small core particle(s) and the large core particle(s) can be used in proportions such that the weight ratio of the small core particle(s) to the large core particle(s) is from 10:90 to 90:10, preferably from 20:80 to 80:20, and more preferably from 30:70 to 70:30.

In a particular embodiment, the weight ratio of the small core particle(s)/the large core particle(s)/the inorganic or organic particulate solid UV filter(s) may be from 20:50:30 to 50:20:30, preferably from 35:15:50 to 15:35:50, and more preferably from 10:20:70 to 20:10:70.

In a preferred embodiment, the weight ratio of the small core particle(s)/the large core particle(s)/the inorganic or organic particulate solid UV filter(s) may be from 50:20:30 or 35:15:50.

The mechanochemical fusion process, in particular the hybridizer process, enables to provide a composite pigment in which small core particles are at least in part covered by at least one layer comprising at least one inorganic or organic particulate solid UV filter, and optionally at least one large core particle and/or at least one coloring pigment and/or at least one additional UV filter. The surface of the large core particles may also be at least in part covered by at least one layer comprising at least one selected from the group consisting of organic particulate solid UV filters, inorganic solid UV filters, coloring pigments and additional UV filters.

Furthermore, the mechanochemical fusion process, in particular the hybridizer process, can provide ordered array (e.g., uniform coverage) of inorganic or organic particulate solid UV filter(s), and optionally at least one coloring pigment and/or at least one additional UV filter on small core particles (and possible large core particles as well) and provides strong bonds at the surface of the small (and large) core particle and a coating layer comprising the inorganic or organic particulate solid UV filter(s), and optionally coloring pigment(s) and/or additional LTV filter(s).

If the large core particles are used in combination with the small core particles, according to the present invention, the inorganic or organic particulate solid UV filter, and optionally the additional UV filter and/or the coloring pigment, can be effectively bound on the surface of the small core particles due to the anchor effects by the collision of the large core particles to the small core particles. Therefore, the UV filtering effects, and optionally coloring effects, can be further enhanced.

It should be noted that the mechanochemical fusion process, in particular the hybridizer process, is quite different from other processes using, for example, a beads mill and a jet mill. In fact, a beads mill causes pulverization or aggregation of core particles, and a jet mill causes pulverization of core particles and uniform coating of a core particle by fine particles is difficult to be formed.

If necessary, an additional process for further coating the composite pigments with UV filter(s) and/or coloring material(s) may be performed. As a result of this additional process, the composite pigment according to the present invention may be coated with a further layer comprising UV filter(s) and/or coloring material(s), preferably consisting of UV filter(s) and/or coloring material(s).

The composite pigment, as described above, can be present in the cosmetic composition according to the present invention in an amount ranging from 0.01% to 99% by weight, preferably from 0.1% to 50% by weight, and more preferably from 1% to 30% by weight, relative to the total weight of the composition.

Preferably, the composite pigment can be used in cosmetic compositions to be applied to keratin substances such as skin, hair, and nails, providing superior UV shielding effects, and optionally coloring effects, because the composite pigment can exhibit good UV filtering effects possibly with a transparent or clear appearance and/or good coloring effects such as more transparent or clearer and more bright coloring, without the risk of affecting keratin substances. Furthermore, the composite pigment according to the present invention is easy to be formulated into and can be stabilized in cosmetic compositions.

Since the composite pigment can reduce free particles which have a high friction coefficient such that they do not easily spread on the skin and provide an unpleasant feeling on use, the cosmetic composition according to the present invention has reduced friction, and therefore, can provide the effect of a better smooth feeling on use.

[Plate-Type Filler with Specific Refractive Index]

The cosmetic composition according to the present invention comprises at least one plate-type filler with a refractive index of more than 1.6 and less than 2.2 in an amount of more than 1% by weight relative to the total weight of composition.

It is preferable that the plate-type filler has a refractive index of 1.7 or more, more preferable 1.8 or more, and furthermore preferable 1.9 or more, and even furthermore preferable 2.0 or more.

It is preferable that the plate-type filler has a refractive index of 2.15 or less, more preferable 2.10 or less, and furthermore preferable 2.05 or less.

The plate-type filler may have a particle size from 1 to 15 μm, preferably from 1 to 10 μm.

The particle size are expressed as the mean volume diameter (D[0.5]).

It is preferable that the plate-type filler be selected from the group consisting of boron nitride, barium sulfate, bismuth oxychloride, alumina and composite powders based on titanium oxide and substrate like talc, mica, barium sulfate, boron nitride, bismuth oxychloride, alumina and mixtures thereof.

In a particular embodiment, the plate type filler is a boron nitride.

In a preferred embodiment, the plate type filler is a boron nitride having a particle size between 1 μm and 10 μm, and in particular between 1 and 6 μm.

As examples of commercial products of boron nitride, we may use the following products: PUHP3008 from Saint Gobains Ceramics (mean particle size 6 μm), the PUHP1030L from Saint Gobain Ceramics (mean particle size 3 μm), the Softouch BN CC6058 powder from Momentive Performance Materials (mean particle size 5-15 μm), or mixtures thereof.

The plate-type filler may be present in the cosmetic composition according to the present invention in an amount ranging from 1% to 20% by weight of the composition, and preferably from 2% to 10% by weight relative to the total weight of the composition.

### [Optional Components]

The cosmetic composition according to the present invention may further comprise at least one additional filler and/or at least one oil.

As used herein, the term “filler” should be understood as meaning colorless natural or synthetic particles of any shape which are insoluble in the medium of the composition, whatever the temperature at which the composition is manufactured. Thus, the filler is different from the coloring pigment as described above.

The fillers may be inorganic or organic and of any shape (for instance, platelet, spherical, and oblong shapes) and with any crystallographic form (for example, sheet, cubic, hexagonal, orthorhombic, and the like). Examples of suitable additional fillers include, but are not limited to, talc; mica; silica; kaolin; powders of polyamide such as Nylon®; poly-β-3-alanine powders; polyethylene powders; polyurethane powders, such as the powder formed of hexamethylene diisocyanate and trimethylol hexyllactone copolymer sold under the name Plastic Powder D-400 by Toshiki; the powders formed of tetrafluoroethylene polymers (Teflon®); lauroyllysine; starch; boron nitride; polymeric hollow microspheres, such as microspheres of poly(vinylidene chloride)/acrylonitrile, for example Expancel® (Nobel Industrie), and microspheres of acrylic acid copolymers; silicone resin powders, for example, silsesquioxane powders (for instance, silicone resin powders disclosed in European Patent No. 0 293 795 and Tospearls® from Toshiba); poly(methyl methacrylate) particles; precipitated calcium carbonate; magnesium carbonate; basic magnesium carbonate; hydroxyapatite; hollow silica microspheres; glass microcapsules; ceramic microcapsules; metal soaps derived from organic carboxylic acids comprising from 8 to 22 carbon atoms, for example, from 12 to 18 carbon atoms, such as zinc stearate, magnesium stearate, lithium stearate, zinc laurate, and magnesium myristate; barium sulphate; and mixtures thereof.

The filler may be present in the composition in an amount ranging from 0.1% to 80% by weight, with respect to the total weight of the composition, for example, from 1% to 25% by weight, or from 3% to 15% by weight.

The term “oil” is understood to mean a fatty substance which is liquid at ambient temperature (25° C.).

Use may be made, as oils which can be used in the composition of the invention, for example, of hydrocarbon oils of animal origin, such as perhydrosqualene (or squalane); hydrocarbon oils of vegetable origin, such as triglycerides of caprylic/capric acids, for example those marketed by Stearineries Dubois or those marketed under the trademarks Miglyol 810, 812 and 818 by Dynamit Nobel, or oils of vegetable origin, for example sunflower, maize, soybean, cucumber, grape seed, sesame, hazelnut, apricot, macadamia, arara, coriander, castor, avocado or jojoba oil or shea butter oil; synthetic oils; silicone oils, such as volatile or non-volatile polymethylsiloxanes (PDMSs) comprising a linear or cyclic silicone chain which are liquid or paste at ambient temperature; fluorinated oils, such as those which are partially hydrocarbon and/or silicone, for example those described in JP-A-2-295912; ethers, such as dicaprylyl ether (CTFA name); and esters, such as benzoate C₁₂-C₁₅ fatty alcohols (Finsolv TN from Finetex); arylalkyl benzoate derivatives, such as 2-phenylethyl benzoate (X-Tend 226 from ISP); amidated oils, such as isopropyl N-lauroylsarcosinate (Eldew SL-205 from Ajinomoto), and their mixtures.

The oily phase can also comprise one or more fatty substances selected, for example, from fatty alcohols (cetyl alcohol, stearyl alcohol, cetearyl alcohol), fatty acids (stearic acid) or waxes (paraffin wax, polyethylene waxes, carnauba wax, beeswax). The oily phase can comprise lipophilic gelling agents, surfactants or also organic or inorganic particles.

The oily phase can preferably represent from 1 to 70% of oil by weight, with respect to the total weight of the composition.

The composition according to the present invention may further comprise at least one additional conventional cosmetic ingredient which may be chosen, for example, from hydrophilic or lipophilic gelling and/or thickening agents, surfactants, antioxidants, fragrances, preservatives, neutralizing agents, sunscreens, vitamins, moisturizing agents, self-tanning compounds, antiwrinkle active agents, emollients, hydrophilic or lipophilic active agents, agents for combating pollution and/or free radicals, sequestering agents, film-forming agents, dermo-decontracting active agents, soothing agents, agents which stimulate the synthesis of dermal or epidermal macromolecules and/or which prevent their decomposition, antiglycation agents, agents which combat irritation, desquamating agents, depigmenting agents, antipigmenting agents, propigmenting agents, NO-synthase inhibitors, agents which stimulate the proliferation of fibroblasts and/or keratinocytes and/or the differentiation of keratinocytes, agents which act on microcirculation, agents which act on energy metabolism of the cells, healing agents, and mixtures thereof.

In a particular embodiment, the cosmetic composition of the present invention comprises a low amount of additional coloring pigments. As the additional coloring pigments, mention may be made of those to be used as the coloring pigments listed for the coating layer of the small or large particle.

The cosmetic composition according to the present invention may comprise from 0 to 5% of additional coloring pigments relative to the total weight of the composition.

In a particular embodiment, the cosmetic composition according to the present invention comprises from 0 to 3% of additional coloring pigments relative to the total weight of the composition.

The composition according to the present invention may be in various forms, for example, suspensions, dispersions, solutions, gels, emulsions, such as oil-in-water (O/W), water-in-oil (W/O), and multiple (e.g., W/O/W, polyol/O/W, and O/W/O) emulsions, creams, foams, sticks, dispersions of vesicles, for instance, of ionic and/or nonionic lipids, two-phase and multi-phase lotions, sprays, powders, and pastes. The composition may be anhydrous, for example, it can be an anhydrous paste or stick. The composition may also be a leave-in composition.

According to one embodiment, the cosmetic composition according to the present invention may be in the form of a powdery composition or a liquid or solid composition, such as an oily-solid cosmetic composition or an anhydrous composition.

In particular, the powdery cosmetic composition according to the present invention can have reduced friction which provides a smooth feeling to use, and can have good compactability which provides high stability against physical impact, due to the inclusion of the composite pigment according to the present invention.

Furthermore, the powdery cosmetic composition according to the present invention can show preferable cosmetic effects such as good fitting to the skin, homogeneous appearance, hiding the color of the skin, hiding the pores and lines on the skin, making the pores and lines on the skin less remarkable, and matt appearance, due to the inclusion of the combination of the above composite pigment, the organopolysiloxane elastomer and the specific oil absorbing agent with a specific amount, according to the present invention.

On the other hand, the liquid cosmetic composition according to the present invention can also show good skin pore hiding effects and good visual optical effects such as matt and haze effects, due to the inclusion of the combination of the above composite pigment, the organopolysiloxane elastomer and the specific oil absorbing agent with a specific amount, according to the present invention.

In particular, the powdery and liquid cosmetic composition according to the present invention has better UV filtering effects, and optionally better coloring effects, in addition to reduce the risk of fine particles of inorganic solid UV filter(s) and optional coloring pigment(s) penetrating into the skin via pores on the skin.

According to another embodiment, the cosmetic composition according to the present invention may be in the form of, for example, a compact powder, a lotion, a serum, a milk, a cream, a base foundation, an undercoat, a make-up base coat, a foundation, a face powder, cheek rouge, a lipstick, a lip cream, an eye shadow, an eyeliner, a loose powder, a concealer, a nail coat, mascara, a sunscreen and the like.

According to another embodiment, the cosmetic composition according to the present invention may be in the form of a foam.

According to this embodiment, the cosmetic composition according to the present invention can be packaged in a foam dispenser. It can involve either products referred to as “aerosols” dispensed from a pressurized container by means of a propellant gas and thus forming a foam at the time of their dispensing, or products dispensed from a container by means of a mechanical pump connected to a dispensing head where the passage of the cosmetic composition through the dispensing head transforms it into a foam in the area of the outlet orifice of such a head at the latest.

According to a first variant, the dispenser can be an aerosol furthermore containing the cosmetic composition according to the present invention; and a propellant gas. For the purposes of the invention, the term “propellant” means any compound that is gaseous at a temperature of 20° C. and at atmospheric pressure, and that can be stored under pressure in liquid or gaseous form in an aerosol container. The propellant may be chosen from optionally halogenated volatile hydrocarbons, such as n-butane, propane, isobutane, pentane or a halogenated hydrocarbon, and mixtures thereof. Carbon dioxide, nitrous oxide, dimethyl ether (DME), nitrogen or compressed air may also be used as propellant. Mixtures of propellants may also be used. Dimethyl ether and/or non-halogenated volatile hydrocarbons are preferably used.

The propellant gas which can be used may be chosen among the previously mentioned gases and in particular among carbon dioxide, nitrogen, nitrogen oxide, dimethyl ether, volatile hydrocarbons such as butane, isobutane, propane and pentane, and mixtures thereof.

According to another variant, the cosmetic composition according to the present invention can be in a “pump bottle” type foam dispenser. These dispensers include a dispensing head for delivering the cosmetic composition, a pump and a plunger tube for transferring the cosmetic composition from the container, into the head, for dispensing the product. The foam is formed by forcing the cosmetic composition to pass through a material including a porous substance such as a sintered material, a filtering grid of plastic or metal, or similar structures.

Such dispensers are known to a person skilled in the art and are described in the patents: U.S. Pat. No. 3,709,437 (Wright), U.S. Pat. No. 3,937,364 (Wright), U.S. Pat. No. 4,022,351 (Wright), US patent 4,1147,306 (Bennett), U.S. Pat. No. 4,184,615 (Wright), U.S. Pat. No. 4,598,862 (Rice), U.S. Pat. No. 4,615,467 (Grogan et al.), and U.S. Pat. No. 5,364,031 (Tamiguchi et al.).

### [Cosmetic Process]

Another aspect of the present invention is a cosmetic process using the cosmetic composition according to the present invention as explained above.

The cosmetic process according to the present invention includes at least a step of applying the cosmetic composition according to the present invention onto the skin.

The cosmetic process according to the present invention can improve optical brightness and/or homogeneity of the skin, as well as UV filtration effects, without deteriorating other cosmetic effects such as smoothening effects.

It is to be understood that a person skilled in the art can choose the appropriate presentation form, as well as its method of preparation, on the basis of his/her general knowledge, taking into account the nature of the constituents used, for example, their solubility in the vehicle, and the application envisaged for the composition.

### EXAMPLES

The present invention will be described in more detail by way of examples, which however should not be construed as limiting the scope of the present invention.

### Pigment Examples

The components shown in Table 1 were subjected to a hybridizer process using a Hybridizer equipped with a high speed rotor having a plurality of blades in a chamber in dry conditions, marketed by Nara Machinery Co., Ltd. in Japan to obtain a composite pigment.

In detail, for each of Pigment Examples 1 to 4, the components shown in Table 1 were mixed at the mixing ratio (the numerals in Table 1 are based on parts by weight) shown in Table 1 in a plastic bag by hand shaking for a short period of time. The mixture was put in the Hybridizer, and the rotor was revolved at 8,000 rpm (100 m/s linear velocity) for 3 minutes to obtain the composite pigments according to Pigment Examples 1 to 4.

As Control, a marketed composite pigment with silica and titanium dioxide (SUNSIL-Tⁱⁿ50 marketed by Sunjin Chemical Co., Ltd.) was used. In this composite pigment, titanium oxide fine particles are distributed in a solid silica particle. The particle size of the composite pigment according to Control was 4 μm.

### (UV Absorbance Evaluation)

Absorbance of UV waves of each of the composite pigments according to Pigment Examples 1 to 4 and Control was measured by use of a UV/VIS spectrophotometer type V-550 (JASCO, Japan) as follows.

A solvent was prepared by mixing isododecane and polyhydroxystearic acid such that the concentration of polyhydroxystearic acid was 3 wt %.

Each of the composite pigments according to Pigment Examples 1 to 4 and Control was dispersed in the above solvent by using ultrasonic waves for 1 minute to obtain a sample, such that the concentration of the composite pigment in the sample was 0.1 wt %. If agglomerates were still present, the ultrasonic treatment was repeated.

The obtained sample was put into a quartz cell having a 2 mm light pathway. The UV absorbance of the sample in the wavelength of from 280 to 400 nm was measured by use of a UV/VIS spectrophotometer type V-550 (JASCO, Japan).

The results are shown in Table 1 in the column of “UV*”.

It is clear that the UV absorbance of the composite pigments according to Pigment Examples 1 to 4 is enhanced.

Since a relatively large amount of TiO₂ is used in Pigment Examples 1 and 3, the UV absorbance value of Examples 1 and 3 is higher than that of Examples 2 and 4, respectively. Since TiO₂ nano particles easily form aggregations which are difficult to show good UV absorption, it is surprising to observe that a relatively large amount of TiO₂ can exert higher UV absorbance for the composite pigment according to Examples 1 and 3.

TABLE 1

| | Small Hollow | | | | | |
|---|---|---|---|---|---|---|
| | Core | Small Solid | Large | | | |
| | Styrene/Acrylate | Core | Core | | UV filter | |
| | Copolymer | PMMA(1) | PMMA(2) | Nylon 12 | TiO₂ | |
| Particle Size | 350 nm | 350 nm | 6 μm | 5 μm | 15 nm | UV* |
| Pigment Ex. 1 | 35 | — | 15 | — | 50 | 129 |
| Pigment Ex. 2 | 50 | — | 20 | — | 30 | 102 |
| Pigment Ex. 3 | — | 35 | — | 15 | 50 | 148 |
| Pigment Ex. 4 | — | 35 | — | 35 | 30 | 72 |
| Control | — | — | — | — | — | 19 |

Syrene/Acrylate Copolymer: Sunspheres marketed by Rohm and Haas
PMMA(1): MP-2200 marketed by Soken in Japan
PMMA(2): MR-7GC marketed by Soken in Japan
Nylon 12: SP-500 marketed by Toray in Japan
TiO₂: MT-100 TV marketed by Tayca in Japan
UV*: UV absorbance in the wavelength region from 280 to 400 nm

### Examples 1 and 2, and Comparative Examples 1 to 5

A cosmetic base was prepared by mixing the components shown in Tables 2 and 3. Unless otherwise mentioned, the values in Tables 2 and 3 are expressed as % by weight relative to the total weight of the composition.

TABLE 2

| | | | | Comp. |
|---|---|---|---|---|
| Phase | Component | Ex. 1 | Ex. 2 | Ex. 1 |
| A | Water | 25.00 | 25.00 | 25.00 |
| | Disodium EDTA | 0.10 | 0.10 | 0.10 |
| | Phenoxyethanol | 0.30 | 0.30 | 0.30 |
| | Glycerin | 7.00 | 7.00 | 7.00 |
| | Potassium Cetyl Phosphate | 0.05 | 0.05 | 0.05 |
| | Hydrogenated Lecithin | 0.30 | 0.30 | 0.30 |
| | Sucrose Stearate | 3.00 | 3.00 | 3.00 |
| | Salicylic Acid | 0.20 | 0.20 | 0.20 |
| B | Stearic Acid | 0.10 | 0.10 | 0.10 |
| | Caprylyl Glycol | 0.30 | 0.30 | 0.30 |
| | Caprylic/Capric Triglyceride | 2.50 | 2.50 | 2.50 |
| | Dicaprylyl Carbonate | 2.00 | 2.00 | 2.00 |
| | Tocopheryl Acetate | 0.10 | 0.10 | 0.10 |
| C | Caprylic/Capric Triglyceride | 2.00 | 2.00 | 2.00 |
| | Xanthan Gum | 0.25 | 0.25 | 0.25 |
| | Acrylates/C₁₀₋₃₀ Alkyl Acrylate | 0.10 | 0.10 | 0.10 |
| | Crosspolymer | | | |
| | Carbomer | 0.20 | 0.20 | 0.20 |
| D | Water | 17.05 | 19.05 | 19.05 |
| E | Pigment Example 2 | 2.00 | — | 4.00 |
| | Pigment Example 3 | | 2.00 | — |
| | Boron Nitride* | 4.00 | 2.00 | — |
| F | Water | 20.00 | 20.00 | 20.00 |
| | 3-O-Ethyl Ascorbic Acid | 1.00 | 1.00 | 1.00 |
| | Citric Acid | 0.10 | 0.10 | 0.10 |
| | Sodium Citrate | 0.10 | 0.10 | 0.10 |
| G | Hydroxypropyl tetrahydropyrantriol | 9.00 | 9.00 | 9.00 |
| | Citric Acid | 0.15 | 0.15 | 0.15 |
| | Fragrance | 0.10 | 0.10 | 0.10 |
| | Alcohol (denat.) | 3.00 | 3.00 | 3.00 |

| | | | |
|---|---|---|---|
| Total | 100.00 | 100.00 | 100.00 |

Boron Nitride*: PUHP 1030L-72727 with refractive index of 2.17 marketed by Saint Gobain Ceramics

TABLE 3

| | | Comp. | Comp. | Comp. | Comp. |
|---|---|---|---|---|---|
| Phase | Component | Ex. 2 | Ex. 3 | Ex. 4 | Ex. 5 |
| A | Water | 25.00 | 25.00 | 25.00 | 25.00 |
| | Disodium EDTA | 0.10 | 0.10 | 0.10 | 0.10 |
| | Phenoxyethanol | 0.30 | 0.30 | 0.30 | 0.30 |
| | Glycerin | 7.00 | 7.00 | 7.00 | 7.00 |
| | Potassium Cetyl Phosphate | 0.05 | 0.05 | 0.05 | 0.05 |
| | Hydrogenated Lecithin | 0.30 | 0.30 | 0.30 | 0.30 |
| | Sucrose Stearate | 3.00 | 3.00 | 3.00 | 3.00 |
| | Salicylic Acid | 0.20 | 0.20 | 0.20 | 0.20 |
| B | Stearic Acid | 0.10 | 0.10 | 0.10 | 0.10 |
| | Caprylyl Glycol | 0.30 | 0.30 | 0.30 | 0.30 |
| | Caprylic/Capric | 2.50 | 2.50 | 2.50 | 2.50 |
| | Triglyceride | | | | |
| | Dicaprylyl Carbonate | 2.00 | 2.00 | 2.00 | 2.00 |
| | Tocopheryl Acetate | 0.10 | 0.10 | 0.10 | 0.10 |
| C | Caprylic/Capric | 2.00 | 2.00 | 2.00 | 2.00 |
| | Triglyceride | | | | |
| | Xanthan Gum | 0.25 | 0.25 | 0.25 | 0.25 |
| | Acrylates/C₁₀₋₃₀ Alkyl | 0.10 | 0.10 | 0.10 | 0.10 |
| | Acrylate Crosspolymer | | | | |
| | Carbomer | 0.20 | 0.20 | 0.20 | 0.20 |
| D | Water | 19.05 | 19.05 | 19.05 | 17.05 |
| E | Pigment Example 2 | — | 2 | — | 2 |
| | Pigment Example 3 | — | — | 2 | — |
| | Boron Nitride* | 2 | — | — | — |
| | TiO₂** | 2 | — | — | — |
| | Talc*** | — | — | 2 | 4 |
| | White Pearl**** | — | 2 | — | — |
| F | Water | 20.00 | 20.00 | 20.00 | 20.00 |
| | 3-O-Ethyl Ascorbic Acid | 1.00 | 1.00 | 1.00 | 1.00 |
| | Citric Acid | 0.10 | 0.10 | 0.10. | 0.10 |
| | Sodium Citrate | 0.10 | 0.10 | 0.10 | 0.10 |
| G | Hydroxypropyl | 9.00 | 9.00 | 9.00 | 9.00 |
| | tetrahydropyrantriol | | | | |
| | Citric Acid | 0.15 | 0.15 | 0.15 | 0.15 |
| | Fragrance | 0.10 | 0.10 | 0.10 | 0.10 |
| | Alcohol (denat.) | 3.00 | 3.00 | 3.00 | 3.00 |

| | | | | |
|---|---|---|---|---|
| Total | 100.00 | 100.00 | 100.00 | 100.00 |

Boron Nitride*: PUHP 1030L-72727 with refractive index of 2.17 marketed by Saint Gobain Ceramics
TiO₂**: NAI-TAO-77891 with refractive index of 2.72 marketed by Miyoshi Kasei in Japan
Talc***: LUZENAC PHARMA M with refractive index of 1.57 marketed by Luzenac
White Pearl****: TiO₂ coated Mica (KTZ FINE WHITE) with refractive index of 2.52 marketed by Taizhu in China

The components in Phases A to D were mixed together in a container with a magnetic stirrer at a temperature of from 60 to 80° C. Then, the components in Phase E were added to the mixture, and stirred. Next, the components in Phases F and G were added to the mixture, and stirred. Lastly, the mixture obtained was homogenized to obtain the cosmetic base according to Examples 1 and 2 as well as Comparative Examples 1 to 5.

### (Sensory Evaluation)

Each of the cosmetic bases according to Examples 1 and 2 as well as Comparative Examples 1 to 5, in an amount of 0.1 g, was applied to the skin of 6 panelists, and the cosmetic effects (i.e., skin brightening effect and color homogeneity) of each cosmetic base was evaluated by experts and compared.

Specifically, the cosmetic base according to Example 1 or 2 was applied to half of the face, and the cosmetic base according to any of the cosmetic bases according to Comparative Examples 1 to 5 was applied to the other half of the face. The cosmetic effects of the former were compared to those of the latter. The evaluations by experts on the 6 panelists were averaged. The results are shown in Table 4.

### (Stability)

Each of the cosmetic bases according to Examples 1 and 2 as well as Comparative Examples 1 to 5, was subjected to centrifugation using a Biofuge Stratos (Heraeus Co) following the recommendations of the supplier. 1 g of each cosmetic base was filled into a tube with an inner volume of 15 ml, and centrifuged at a temperature of 25° C. with a relative centrifugal force of 900 g (2743 rpm), and for 60 minutes. The phase separation (sedimentation of particles in bottom of tube) of the cosmetic base was evaluated by experts. The evaluations by 6 panelists were averaged. The results are shown in Table 4.

TABLE 4

| | Ex. | Ex. | Comp. | Comp. | Comp. | Comp. | Comp. |
|---|---|---|---|---|---|---|---|
| | 1 | 2 | Ex. 1 | Ex. 2 | Ex. 3 | Ex. 4 | Ex. 5 |
| Skin Bright- | Very | Good | Very | Good | Good | Very | Very |
| ening | Good | | Good | | | Bad | Bad |
| Color | Very | Good | Very | Very | Very | Good | Good |
| Homogeneity | good | | Bad | Bad | Bad | | |
| Stability | Good | Good | Bad | Bad | Very | Bad | Good |
| | | | | | Bad | | |

Only the cosmetic composition according to Examples 1 and 2 showed a good balance of skin brightening, color homogeneity and stability.

The above results demonstrate that the cosmetic composition according to Examples 1 and 2 based on a combination of the composite pigment and the plate-type filler with a refractive index of more than 1.6 and less than 2.2 can provide improved optical cosmetic effects and stability, and that other combinations cannot provide such improved optical cosmetic effects and stability.

### Example 3

A TWC foundation was prepared by mixing the components shown in Table 5. Unless otherwise mentioned, the values in Table 5 are expressed as % by weight relative to the total weight of the composition.

TABLE 5

| Ingredient | Wt % |
|---|---|
| Talc/Dimethicone | 42.47 |
| Mica | 15.00 |
| Synthetic fluorphlogopite/Hydrogen methicone | 3.00 |
| Nylon-12 | 6.00 |
| Vinyl dimethicone/Methicone silsesquioxane | 6.00 |
| Titanium dioxide/Lauroyl lysine | 5.00 |
| Pigment Example 1 | 5.00 |
| Boron nitride (PUHP 1030L-72727 with refractive | 4.00 |
| index of 2.17 marketed by Saint Gobain Ceramics) | |
| Yellow Iron oxides | 1.88 |
| Black Iron oxides | 0.33 |
| Red Iron oxides | 0.62 |
| Caprylic/Capric triglyceride | 2.00 |
| Dimethicone | 5.30 |
| Ethylhexyl methoxycinnamate | 3.00 |
| Caprylyl glycol | 0.20 |
| Ethylhexyl glycerin | 0.20 |
| Total | 100 |

### Example 4

An aerosol foam was prepared by mixing the components shown in Table 6. Unless otherwise mentioned, the values in Table 6 are expressed as % by weight relative to the total weight of the composition.

TABLE 6

| Ingredient | Wt % |
|---|---|
| Titanium dioxide (and) silica (and) aluminum hydroxide | 5 |
| (and) alginic acid | |
| Talc | 1 |
| Boron nitride | 4 |
| Pigment Example 2 | 3 |
| Silica (and) methicone | 1.9 |
| Mica (and) titanium oxide | 1.425 |
| Yellow iron oxide (and) isopropyl titanium triisostearate | 0.1425 |
| Red iron oxide (and) isopropyl titanium triisostearate | 0.038 |
| Black iron oxide (and) isopropyl titanium triisostearate | 0.0285 |
| Calcium carbonate | 1.9 |
| Ethylhexyl methoxycinnamate | 7.125 |
| Octocrylene | 2.85 |
| Ethylhexyl salicylate | 4.7025 |
| p-Anisic acid | 0.266 |
| Water | 46.166 |
| Sea water | 7.1725 |
| Dipotassium glycyrrhizate | 0.19 |
| Sodium hyaluronate | 0.0095 |
| Betaine | 0.475 |
| PEG/PPG/polybutylene glycol/8/5/3/glecerin | 2.85 |
| Ethylhexylglycerin | 0.285 |
| Caprylyl glycol | 0.475 |
| Butylene glycol | 1.9 |
| Alcohol | 0.665 |
| PEG-12 dimethicone | 0.95 |
| Phenoxyethanol | 0.3325 |
| Tocopheryl acetate | 0.0095 |
| Fragrance | 0.0475 |
| Propylene glycol (and) hexylene glycol (and) | 0.095 |
| Hamamelis Virginiana (Witch Hazel) extract | |
| Ethane | 0.005 |
| Propane | 1.16 |
| Isobutane | 1.12 |
| Butane | 2.67 |
| Pentane | 0.045 |
| Total | 100 |

### Example 5

A loose powder was prepared by mixing the components shown in Table 7. Unless otherwise mentioned, the values in Table 7 are expressed as % by weight relative to the total weight of the composition.

TABLE 7

| Ingredient | Wt % |
|---|---|
| Talc | 55.85 |
| Boron nitride | 10.00 |
| Mica | 10.00 |
| Aluminum starch octenylsuccinate | 10.00 |
| Pigment Example 2 | 10.00 |
| Iron oxide | 0.15 |
| Magnesium stearate | 2.00 |
| Isocetyl stearate | 1.00 |
| Dimethicone | 1.00 |
| Total | 100 |

1-17. (canceled)

18. A cosmetic composition comprising:
(i) at least one composite pigment comprising at least one small particle with a mean particle size ranging from about 100 nm to about 1 μm;
wherein the surface of the small particle is at least partially covered with at least one coating layer, and
wherein the at least one coating layer comprises at least one inorganic or organic particulate solid UV filter, and optionally at least one coloring agent; and
(ii) at least one plate-type filler with a refractive index ranging from about 1.6 to about 2.2,
wherein the at least one plate-type filler is present in an amount greater than about 1% by weight, relative to the total weight of composition.

19. The cosmetic composition of claim 18, wherein the small particle is chosen from hollow particles.

20. The cosmetic composition of claim 18, wherein the at least one composite pigment further comprises at least one large particle with a mean particle size greater than about 2 μm;
wherein the surface of the large particle is optionally at least partially covered with at least one coating layer; and
wherein the coating layer comprises at least one of:
(i) at least one inorganic particulate solid UV filter;
(ii) at least one organic particulate solid UV filter or
(iii) at least one coloring pigment.

21. The cosmetic composition of claim 20, wherein the at least one coating layer on at least one of the small particles and/or large particles has a thickness ranging from about 1 nm to about 50 nm.

22. The cosmetic composition of claim 20, wherein the coating layer of at least one of the small particles and/or large particle has an inorganic solid UV filter chosen from silicon carbide, metal oxides, and mixtures thereof.

23. The cosmetic composition of claim 22, wherein the inorganic solid UV filter is titanium dioxide.

24. The cosmetic composition of claim 20, wherein the inorganic solid UV filter has a mean particle size ranging from about 1 nm to about 50 nm.

25. The cosmetic composition of claim 20, wherein the at least one small particle or the at least one large particle comprises poly(meth)acrylates, polyamides, silicones, polyurethanes, polyethylenes, polypropylenes, polystyrenes, copolystyrenes, polyhydroxyalkanoates, polycaprolactams, poly(butylene) succinates, polysaccharides, polypeptides, polyvinyl alcohols, polyvinyl resins, fluoropolymers, waxes, amidosulfonic acid polyvalent metal salts, acylated amino acids, and mixtures thereof.

26. The cosmetic composition of claim 20, wherein the at least one small particle comprises at least one organic polymer chosen from copolystyrene polymers.

27. The cosmetic composition of claim 26, wherein the at least one copolystyrene polymer is chosen from styrene/acrylate copolymers and cross-linked styrene/methyl methacrylate copolymers.

28. The cosmetic composition of claim 20, wherein:
the at least one small particle comprises at least one copolystyrene polymer;
the at least one large particle comprises at least one poly(meth)acrylate polymer; and
the small and large particles are at least partially covered with at least one coating layer comprising at least one metal oxide.

29. The cosmetic composition of claim 20, wherein the at least one plate-type filler is present in the composition in an amount ranging from about 1% to about 20% by weight, relative to the total weight of the composition.

30. The cosmetic composition of claim 29, wherein the at least one plate-type filler is present in the composition in an amount ranging from about 2% to about 10% by weight, relative to the total weight of the composition.

31. The cosmetic composition of claim 20, wherein the at least one plate-type filler is chosen from boron nitride, barium sulfate, bismuth oxychloride, alumina, composite powders based on titanium oxide and a substrate, and mixtures thereof.

32. The cosmetic composition of claim 20, wherein the at least one plate-type filler has a particle size ranging from about 1 to about 15 μm.

33. The cosmetic composition of claim 32, wherein the at least one plate-type filler has a particle size ranging from about 1 to about 10 μm.

34. The cosmetic composition of claim 20, wherein the cosmetic composition is in the form of a liquid, powder, or aerosol foam.

35. A method for improving at least one property chosen from optical brightness and/or homogeneity of the skin, comprising:
applying to the skin at least one layer of a cosmetic composition comprising:
(i) at least one composite pigment comprising at least one small particle with a mean particle size ranging from about 100 nm to about 1 μm;
wherein the surface of the small particle is at least partially covered with at least one coating layer, and
wherein at the at least one coating layer comprises at least one inorganic or organic particulate solid UV filter, and optionally at least one coloring agent; and
(ii) at least one plate-type filler with a refractive index ranging from about 1.6 to about 2.2,
wherein the at least one plate-type filler is present in an amount greater than about 1% by weight, relative to the total weight of composition.

36. The method of claim 35, wherein the composite pigment further comprises at least one large particle with a mean particle size greater than about 2 μm;
wherein the surface of the large particle is optionally at least partially covered with at least one coating layer; and
wherein the coating layer comprises at least one of:
(i) at least one inorganic particulate solid UV filter;
(ii) at least one organic particulate solid UV filter; or
(iii) at least one coloring pigment.

37. The method according to claim 36, wherein:
the at least one small particle comprises at least one copolystyrene polymer;
the at least one large particle comprises at least one poly(meth)acrylate polymer; and
the small and large particles are at least partially covered with at least one coating layer comprising at least one metal oxide.
